(19) United States
(12) Patent Application Publication
(10) Pub. No.: US 2013/0261671 A1
(43) Pub. Date: Oct. 3, 2013

(54) DEVICE FOR BONE REGENERATION AND BONE DISTRACTION

(71) Applicant: Domonkos Horvath, Jestetten (DE)

(72) Inventor: Domonkos Horvath, Jestetten (DE)

(73) Assignee: CELGEN AG, Zug (CH)

(21) Appl. No.: 13/992,325

(22) PCT Filed: Dec. 7, 2011
(86) PCT No.: PCT/EP2011/006135

§ 371 Date: Jun. 7, 2013

(30) Foreign Application Priority Data
Dec. 10, 2010 (DE) 102010055433.2

(51) Int. Cl. A61B 17/66; A61B 17/86

(52) U.S. Cl. CPC A61B 17/663; A61B 17/8685
USPC 606/282; 606/306

(57) ABSTRACT

The present invention relates to bone screws, membranes and devices for regenerating a bone, in particular by way of callus distraction, notably in the jaw region, to the use of the bone screws, membranes and devices for callus distraction, and to methods for callus distraction.

The present invention relates to bone screws, membranes and devices for regenerating a bone, in particular by way of callus distraction, notably in the jaw region, to the use of the bone screws, membranes and devices for callus distraction, and to methods for callus distraction.

Bone losses are currently generally filled in with bone substitute material or with autogenous or allogenic bone.

From a biological view, an autologous spongiosa transplant is the best substitute material for bone. However, such transplants are only available to a limited extent and exhibit a high resorption rate after transplantation.

The materials and techniques employed in the prior art frequently provide inadequate bone quality, so that implants, for example, are not rigidly anchored in the beds. Additionally, the bone substitute is frequently not sufficiently vascularized, and as a result the risk of infection is increased. Methods according to the prior art often also employ growth factors, which significantly increase the costs of the procedures.

Instead of using a bone substitute, lacking bone substance can also be partially filled in by way of bone regeneration. Segmental osseous discontinuity on long bones can thus be treated by way of distraction osteogenesis.

This callus distraction has been known for more than one hundred years. The most important biological stimulus for osteogenesis is mechanical stress. Piezoelectrical forces are released in the process, which activate osteoblasts and osteoclasts. Distraction osteogenesis induces new bone formation by triggering biological growth stimuli by slowly separating bone segments. This method achieves the direct formation of woven bone by way of distraction. Defined tensile stress during bone generation is essential. If such defined tensile stress is applied to bone fragments, the mesenchymal tissues in the gap and on the adjoining fragment ends show osteogenetic capacity. If sufficient vascular potency exists, progressive distraction results in metaplasia of the organized hematoma, also referred to as a blood clot, in a zone of longitudinally arranged, fibrous tissue, which under optimal external and internal conditions can convert directly into woven bone. However, an aggravating factor is that the bone tissue is subject to highly complex control during regeneration.

WO 01/91663 A1 and U.S. Pat. No. 5,980,252 describe devices and methods for callus distraction by way of artificial interfaces, for example membranes. The membranes used there are flat plates or flat small plates, which are usually made of metal, for example titanium. When these plates or small plates are moved for distraction of the bone, the edges and the lateral faces thereof, which form the height of the membrane, graze the neighboring tissue. This results in irritation and further injury of the neighboring tissue, whereby healing may be worsened.

In addition, these flat plates can only be used to distract smaller regions of a bone section that is not planar, which is to say fiat, for example of a jaw, because otherwise the membrane is not evenly seated against the bone.

For use with bone distraction, the membranes from the prior art in particular require distraction devices, which are complex, and consequently large. The distraction devices must be fixed to teeth located next to the bone defect to be treated. If these teeth are not present or porous, bone distraction is not possible. The distraction devices according to the prior art also only allow distraction leading perpendicularly away from the alveolar crest.

The technical problem underlying the present invention is to provide devices that make it possible to carry out bone regeneration methods that overcome the drawbacks of the prior art.

The technical problem underlying the present invention is also to provide devices that make it possible to carry out space-saving bone regeneration methods.

The technical problem underlying the present invention is also to provide simplified devices that make it possible to carry out bone regeneration methods, notably in the jaw region, but also on other bones.

The technical problem underlying the present invention is also to provide devices that make it possible to carry out large-surface-area bone regeneration methods.

The technical problem underlying the present invention is also to provide devices that make it possible to carry out bone regeneration methods in the jaw region if the bone defect is not delimited by teeth, or if the teeth delimiting the bone defect are not suitable for fixing a distraction device.

The technical problem underlying the present invention is also to provide devices that make it possible to regenerate bone sections that are not planar, which is to say flat, for example an alveolar ridge, or defects in a long bone.

The technical problem underlying the present invention is also to provide devices that make it possible to regenerate atrophied bone defects, and more particularly alveolar ridges.

The technical problem underlying the present invention is also to provide devices that make it possible to carry out periodontal regenerations, especially in a simple manner.

The present invention solves the underlying technical problem, in particular, by providing bone screws, membranes, in particular distraction membranes, devices, methods and uses according to the claims.

The present invention solves the underlying technical problem in particular by providing a bone screw comprising a first screw body part and a second screw body part, wherein the first screw body part has a first external thread for sinking the screw into a bone and a second external thread that is oriented in the opposite direction of the first external thread, wherein the second external thread has a smaller diameter than the first external thread, and wherein a screw body intermediate piece is located between the first and second external threads, wherein the diameter of the screw body intermediate piece is smaller than the diameter of the first external thread and at least equal in size to the diameter of the second external thread, and wherein the transition from the first external thread to the screw body intermediate piece has a chamfer or a rounding, and wherein the second screw body part is a screw nut comprising an internal thread, wherein the internal thread is oriented in the same direction as the second external thread of the first screw body part, and the first and second screw body parts can be detachably connected to each other by way of the second external thread and the internal thread, wherein the second screw body part has a chamfer or a rounding toward the opening of the internal thread, the opening facing the first screw body part when the parts are connected. In the present invention, such a bone screw is referred to as a bone screw of the first embodiment.

A preferred embodiment according to the invention relates to a bone screw comprising a first screw body part and a second screw body part, wherein the first screw body part comprises a first external thread for sinking the screw into a bone and a second external thread that is oriented in the opposite direction of the first external thread, wherein the diameter of the second external thread is smaller than that of the first external thread, and wherein a screw body intermediate piece is located between the first and second external threads, wherein the diameter of the screw body intermediate piece is smaller than the diameter of the first external thread and is at least equal in size to the diameter of the second external thread, and wherein the transition from the first external thread to the screw body intermediate piece has a or is rounded, and wherein the second screw body part is a screw nut comprising an internal thread, wherein the internal thread is oriented in the same direction as the second external thread of the first screw body part, and the first and second screw body parts can be detachably connected to each other by way of the second external thread and the internal thread, wherein the second screw body part has a chamfer or is rounded toward an opening of the internal thread, the opening facing the first screw body part when the parts are connected.

The present invention also solves the underlying technical problem by a bone screw comprising a first screw body part and a second screw body part, wherein the first screw body part comprises a first external thread for sinking the screw into a bone and an internal thread that is oriented in the opposite direction of the first external thread, wherein, at the end of the first screw body part located opposite the tip of the first screw body part, the internal thread extends partially into the first screw body part, and wherein the end of the first screw body part located opposite the tip of the first screw body part is chamfered or rounded, and wherein the second screw body part comprises a second external thread and a screw head that is chamfered or rounded toward the second external thread, wherein the second external thread is oriented in the same direction as the internal thread of the first screw body part, and the first and second screw body parts can be detachably connected to each other by way of the internal thread and the second external thread. In the present invention, such a bone screw is referred to as a bone screw of the second embodiment.

An alternative embodiment according to the invention relates to a bone screw comprising a first screw body part and a second screw body part, wherein the first screw body part comprises a first external thread for sinking the screw into a bone and an internal thread that is oriented in the opposite direction of the first external thread, wherein, at the end of the first screw body part located opposite a tip of the first screw body part, the internal thread extends partially into the first screw body part, and wherein the end of the first screw body part located opposite the tip of the first screw body part is chamfered or rounded, and wherein the second screw body part comprises a second external thread and a screw head that is chamfered or rounded toward the second external thread, wherein the second external thread is oriented in the same direction as the internal thread of the first screw body part, and the first and second screw body parts can be detachably connected to each other by way of the internal thread and the second external thread.

The bone screws of the first and second embodiments are alternative embodiments of an inventive concept, namely providing a bone screw to which a distraction membrane can be secured at various angles. Both embodiments allow the membranes to be secured at various angles by clamping the membrane having a perforation between the first and second screw body parts, wherein the clamping can also allow some play in terms of the mobility of the membrane, so that the membrane, due to the chamfered or rounded regions of the first and second screw body parts, does not necessarily have to be at right angles relative to the bone screw axis, but the membrane can also be clamped between the two screw body parts at different angles. In addition, both the bone screws of the first embodiment and those of the second embodiment allow the bone screws to be slowly unscrewed from a bone, in particular a jaw bone, for callus distraction by way of the associated lifting of a distraction membrane, wherein the membrane remains clamped between the two body parts of the membrane, while the bone screw is able to turn in the perforation of the membrane due to the chamfered or rounded regions of the first and second screw body parts. In addition, when the bone screws of the first and second embodiments are unscrewed from the bone using a tool, for example a regular wrench or an Allen wrench, which engages on the second screw body part, the opposite handedness of the second external thread and of the internal thread as compared to the first external thread in both embodiments causes the second screw body part to be remain rigidly connected to the first screw body part.

The length and dimensioning of the bone screw, and of the first screw body part in particular, correspond to the length and dimensioning of conventional bone screws, in particular those that are intended for use in the jaw region.

The bone screw can be made of any material that is suitable for bone screws. The bone screw can preferably be made of a metal, and more particularly titanium or a titanium alloy.

In one embodiment, a fixation aid, and more particularly a bioresorbable fixation aid, is associated with the bone screw according to the invention. The bioresorbable fixation aid can be a bone screw or a dowel, for example. The bone screw according to the invention can be screwed into the fixation aid by way of the first external thread, especially after the fixation aid has been introduced into a bone, in particular a jaw bone. This is particularly advantageous if the bone substance is of poor quality. In this embodiment using bioresorbable fixation aids, the bone screw according to the invention is preferably intended for single use.

A preferred embodiment is a bone screw according to the invention for single use. However, it is also possible to use a bone screw multiple times.

A preferred embodiment is a bone screw according to the invention for use with a medical indication, and more particularly with a first or a second medical indication. A preferred embodiment is a bone screw according to the invention for medical, in particular surgical, use. A further preferred embodiment is a bone screw according to the invention for surgical use in the jaw region. A further preferred embodiment is a bone screw according to the invention for use with callus distraction, especially in the jaw region.

The first screw body part is a bone fixation element. This is primarily used to fix the bone screw in a bone.

The first external thread can be a thread that is known from the prior art, as it is used to fix a screw in a bone. The first external thread can be right-handed or left-handed.

The second external thread and the internal thread are oriented in the opposite direction of the first external thread. If the first external thread is a right-handed thread, the second external thread and the internal thread are thus left-handed, and conversely.

The first screw body part can comprise a section that is designed for a tool, for example a regular wrench, and more particularly a hexagon wrench or a square wrench, or an Allen wrench, to engage in the section, and the first screw body part can thus be screwed into a bone, for example a jaw bone, using the tool. The section for engaging a tool, in particular a tool wrench, can be polygonal, for example, such as hexagonal or tetragonal or, if the section is situated at the end of the first screw body part located opposite the first external thread, it can have an inside shape for receiving a screwdriver, for example a slotted, Phillips, Pozidriv, Torx, hex key, Robertson, Tri-wing, Torq-set or spanner shape.

A person skilled in the art will know suitable shapes and suitable tools.

In a preferred embodiment, the section for engaging a tool, in particular a tool wrench, is a wrench surface that essentially has four faces, wherein the edges between the four surfaces in particular can be flattened.

For example, on the first screw body part of the bone screw of the first embodiment, the section that is provided in such an embodiment can be located between the first external thread and the second external thread, or at the end of the first screw body part opposite the first external thread, and more particularly can adjoin the second external thread there. For example on the first screw body part of the bone screw of the second embodiment, the section that is provided in such an embodiment can be located between the first external thread and the opening of the Internal thread, which is to say at the head of the first screw body part.

On the first screw body part of the bone screw of the first embodiment, a tool wrench engagement surface is located between the first external thread and the second external thread in a preferred embodiment. On the first screw body part of the bone screw of the second embodiment, a tool wrench engagement surface is located at the head of the first screw body part in a preferred embodiment.

In these embodiments, the tool wrench engagement surface preferably forms part of the first external thread, which is to say the transition between the tool wrench engagement surface and the screw body intermediate piece has a chamfer or a rounding. However, alternatively the screw body intermediate piece can also be designed as a tool wrench engagement surface.

These embodiments have the advantage that the first screw body part can be screwed into a bone by way of a tool wrench only until the tool wrench engagement surface has reached the surface of the bone. By positioning the tool wrench engagement surface, the first screw body part can be prevented from being screwed too far into the bone, in a simple manner.

In a bone screw of the first embodiment, the screw body intermediate piece has a length that corresponds approximately to the thickness of a distraction membrane, and in particular corresponds to, or is slightly longer than, for example 10% than, the thickness of a distraction membrane. If the screw body intermediate piece is also designed as a tool wrench engagement surface, the screw body intermediate piece preferably has a length that allows a tool to be engaged. If, as a result, the length of the screw body intermediate piece should be considerably longer than the thickness of the membrane, this can be easily compensated for by the length and shape of the second screw body part so that a membrane can still be clamped between the first and second screw body parts.

According to the invention, the diameter of the screw body intermediate piece is smaller than the diameter of the first external thread. A membrane having a perforation, for example a round or oval hole or an elongated hole or an angled elongated hole, the diameter of which approximately or exactly corresponds to the diameter of the screw body intermediate piece, can be pushed onto the screw body intermediate piece. However, because of the larger diameter of the first external thread, the membrane can only be attached up to the first external thread. The diameter of the second external thread can be equal in size to, or smaller than, the diameter of the screw body intermediate piece. The membrane having the perforation with a diameter corresponding to the diameter of the screw body intermediate piece can thus be pushed past the second external thread.

The transition from the first external thread to the screw body intermediate piece is chamfered or rounded in a preferred embodiment. In a preferred embodiment, the transition from the first external thread to the screw body intermediate piece has a rounding. The transition is thus preferably rounded, particularly preferably it is convexly rounded. The chamfer or the rounding, in particular a convex rounded region, allows the first screw body part not only to sit perpendicular to a pushed-on membrane, but also to take on an angle different from 90°, which is to say oblique relative to the membrane, because the membrane cannot cant on an edge that is formed by the larger external thread.

On a bone screw of the second embodiment, the first screw body part is a screw comprising an external thread for sinking the screw into a bone and an internal thread having an opening at the screw head. The screw head is preferably not widened, but has the diameter of the first external thread. However, the screw head of the first screw body part of a bone screw of the second embodiment is preferably rounded toward the opening of the internal thread. The internal thread preferably ends in the body of the first screw body part. The internal thread can, for example, have approximately one quarter to approximately one half the length of the first screw body part.

The second screw body part is a membrane fixation element. It is used primarily to fix a membrane, and more particularly to fix a distraction membrane to the bone screw.

On the bone screw of the first embodiment, the second screw body part is a screw nut having an internal thread, wherein the internal thread is oriented in the same direction as the second external thread of the first screw body part, so that the first and second screw body parts can be detachably connected to each other by way of the second external thread and the internal thread. The internal thread of the second screw body part thus not only has the same direction as the second external thread, but preferably also the same diameter and preferably the same flank angle and the same lead.

The internal thread of the second screw body part can extend entirely through the second screw body part, so that the thread forms two openings in the second screw body part. Alternatively and preferably, the internal thread does not extend entirely through the second screw body part. In a preferred embodiment, the internal thread thus protrudes only partially into the second screw body part. For example, the internal thread can have a length of approximately one quarter to approximately three quarters, and in particular approximately one third or approximately one half, of the second screw body part. In this embodiment, the thread thus has only one opening in the second screw body part. The second screw body part is thus a cap nut, in which the internal thread is designed as a blind hole thread. In a preferred embodiment, the internal thread protrudes into the second screw body part only far enough to allow the second screw body part to be screwed onto the first screw body part up to a particular length.

In a preferred embodiment, the second screw body part has a smooth lateral face. The second screw body part can have the shape of a cylinder, cone or truncated cone, for example. The second screw body part is preferably a truncated cone, wherein the edge between the top face and the lateral face is rounded. The top face of the truncated cone preferably comprises the only opening of the internal thread, and the bottom face of the truncated cone comprises a polygonal socket for engaging a tool, in particular an Allen wrench.

In an alternative embodiment, the second screw body part can also comprise an exterior engagement surface for a tool, for example a polygonal head, and more particularly a hexagonal head. This has the advantage that the second screw body part can be shortened.

The preferred embodiment of the second screw body part of the bone screw of the first embodiment in the form of a cylinder, cone, and in particular a truncated cone having a smooth surface and rounded edges, has the advantage that the tissue surrounding or covering the bone to be treated is protected during the distraction because the tissue layers that are located above the distraction membrane, that is, those which come in contact with the second screw body part, are not injured by this part, and more particularly are not injured by sharp edges.

In a preferred embodiment, the second screw body part can only be screwed on to the point where a membrane, which is located between the chamfered or rounded transition from the first external thread to the screw body intermediate piece and the rounded end of the second screw body part comprising the opening of the internal thread, is not yet clamped between the two screw body parts, but has some play, which is to say that the angle between the membrane and the bone screw can change by a certain degree. This has the advantage that the membrane need not be perpendicular to the two screw body parts of the bone screw, but can take on an angle different from 90° relative to the bone screw. However, it is also possible, of course, for the membrane to be clamped without play, in particular if a certain angle is to be maintained, and in particular 90°.

In a preferred embodiment, the second screw body part comprises an engagement section for a tool. The section for engaging a tool, and in particular an Allen wrench, can be polygonal, for example, such as hexagonal or tetragonal or, if the section is preferably located at the end of the second screw body part located opposite the internal thread, it can have an inside shape for receiving a screwdriver, for example a slotted, Phillips, Pozidriv, Torx, hex key, Robertson, Tri-wing, Torq-set or spanner shape. A person skilled in the art will know suitable shapes and suitable tools.

In a preferred embodiment, the section for engaging a tool, and more particularly an Allen wrench, is a substantially polygonal socket having six surfaces.

On the bone screw of the second embodiment, the second screw body part is a screw having an external thread, wherein the external thread is oriented in the same direction as the internal thread of the first screw body part, so that the first and second screw body parts can be detachably connected to each other by way of the internal thread and the second external thread. The second external thread of the second screw body part thus not only has the same direction as the internal thread, but preferably also the same diameter and preferably the same flank angle and the same lead.

On a bone screw of the second embodiment, the second screw body part comprises a screw body intermediate piece between the second external thread and the screw head in a preferred embodiment. The screw body intermediate piece preferably has a length that corresponds approximately to the thickness of a distraction membrane, and in particular corresponds to, or is slightly longer than, for example 10% than, the thickness of a distraction membrane.

According to the invention, the diameter of the screw body intermediate piece is smaller than the diameter of the first external thread. A membrane having a perforation, for example a round or oval hole or an elongated hole, which has a diameter that approximately or exactly corresponds to the diameter of the screw body intermediate piece, can be pushed onto the screw body intermediate piece. However, because of the larger diameter of the first external thread, the membrane can only be attached up to the first external thread. The diameter of the second external thread can be equal in size to, or smaller than, the diameter of the screw body intermediate piece. The membrane having the perforation with a diameter corresponding to the diameter of the screw body intermediate piece can thus be pushed past the second external thread.

The transition from the screw head of the second screw body part to the screw body intermediate piece is chamfered or rounded in a preferred embodiment. In a preferred embodiment, the transition from the screw head to the screw body intermediate piece has a rounding. The transition is thus preferably rounded, particularly preferably it is convexly rounded. The chamfer or the rounding, in particular a convex rounded region, allows the second screw body part not only to sit perpendicular to a pushed-on membrane, but also to take on an angle different from 90°, which is to say oblique relative to the membrane, because the membrane cannot cant on an edge that is formed by the screw head.

In a preferred embodiment, the second screw body part comprises an engagement section for a tool. The section for engaging a tool, and in particular an Allen wrench, can be polygonal, for example, such as hexagonal or tetragonal or, if is preferably located at the end of the second screw body part located opposite the internal thread, the section can have an inside shape for receiving a screwdriver, for example a slotted, Phillips, Pozidriv, Torx, hex key, Robertson, Tri-wing, Torq-set or spanner shape. A person skilled in the art will know suitable shapes and suitable tools.

In a preferred embodiment, the section for engaging a tool, and in particular an Allen wrench, is a substantially polygonal socket having six surfaces.

The present invention also solves the underlying technical problem by a device for bone distraction, comprising at least one bone screw, and more particularly at least two bone screws, and a distraction membrane. In a preferred embodiment, the device comprises at least three, in particular at least four bone screws, particularly four, five, six, seven, eight, nine, ten, eleven, twelve, thirteen or fourteen, or more, bone screws. In a preferred embodiment, the membrane is intended for single use. In a preferred embodiment, the device is intended for single use. In a preferred embodiment, the device is intended for use for callus distraction, in particular in the jaw region.

A preferred embodiment according to the invention relates to a device for bone distraction, comprising at least two bone screws according to the present invention and a distraction membrane, wherein the second external thread of the respective first screw body part of the bone screw of the first embodiment extends through a hole in the membrane and the second external thread of the first screw body part is screwed to the internal thread of the second screw body part of the bone screw so that, at the edge of the hole, the membrane is clamped between the chamfered or rounded transition from the first external thread to the screw body intermediate piece of the first screw body part and the chamfer or rounded region of the second screw body part that is directed toward the internal thread and/or wherein the respective second screw body part of the bone screw of the second embodiment extends through a hole in the membrane and the second external thread of the second screw body part is screwed to the internal thread of the first screw body part of the bone screw so that, at the edge of the hole, the membrane is clamped between the chamfered or rounded end of the first screw body part and the chamfer or rounded region of the screw head of the second screw body part that is directed toward the second external thread. Of course, the at least two screws of the device can be at least two screws of the first embodiment, in particular two screws of the first embodiment, or at least two screws of the second embodiment, in particular two screws of the second embodiment, or at least one screw of the first embodiment, in particular one or two screws of the first embodiment, and at least one screw of the second embodiment, in particular one or two screws of the second embodiment.

The membrane can be a membrane of the prior art or a membrane that is described here, in particular a membrane having perforations. In a preferred embodiment, the bone screws penetrate the membrane through perforations in the membrane. A perforation in the membrane in each case serves as an aperture for a respective bone screw.

In a preferred embodiment, the bone screws are two-piece bone screws, in particular those that can be screwed together by way of an internal thread and an external thread. In a preferred embodiment, the bone screws are bone screws according to the invention. In a preferred embodiment, the bone screws are bone screws according to the invention of the first and/or second embodiments.

In a preferred embodiment, an external thread of a respective screw body part of a bone screw according to the invention extends through a perforation in the membrane, wherein the external thread of the screw body part is screwed to the internal thread of the other screw body part of the bone screw.

The present invention also solves the underlying technical problem by a device for bone distraction, comprising at least one bone screw according to the invention, and more particularly at least two bone screws according to the invention, and a distraction membrane. In a preferred embodiment, the device comprises at least three, in particular at least four bone screws according to the invention, particularly four, five, six, seven, eight, nine, ten, eleven, twelve, thirteen or fourteen, or more, bone screws according to the invention.

The number of bone screws that are present in the device can preferably depend on the size of the membrane of the device. The larger the membrane of the device, the more bone screws can be used so as to assure secure positioning of the membrane over the bone defect to be treated. While as few as two bone screws are sufficient for fixing a small membrane, which can be used, for example, to treat a bone defect in a single tooth gap, in particular devices that comprise at least four bone screws are advantageous, including devices that comprise small membranes. With a membrane that has a basic quadrangular shape, for example, four bone screws can securely position all the corners of the membrane over the bone. Devices comprising larger membranes, which can be used to treat a bone defect in several tooth gaps located next to each other, for example, may comprise more bone screws for secure fixation, for example 6, 8, 10, 12 or 14 bone screws. A device for distracting the majority of an alveolar ridge or an entire alveolar ridge using a horseshoe-shaped membrane can comprise in particular 6, 8 or 10 bone screws.

If the membrane has a plurality of perforations, through which the bone screws can extend, the perforations can advantageously be used to pass through the bone screws when the device is employed in the jaw region, the screws allowing good positioning of the bone screws in suitable locations of the bone, for example in a tooth gap. The positions of the bone screws relative to the membrane can thus be freely selected. In addition, the unused perforations contribute to good blood flow and nutrient exchange between the tissue on the counter-surface of the membrane and the coagel on the contact surface of the membrane.

In an alternative embodiment, the number of perforations of the distraction membrane is at least equal to the number of bone screws of the device.

The bone screws of the device can be bone screws of the first embodiment or bone screws of the second embodiment. It is also possible, of course, for some of the bone screws of the device to be bone screws of the first embodiment and the remaining bone screws to be bone screws of the second embodiment.

In a preferred embodiment of the device, each bone screw is passed through a perforation of the membrane, in particular so that the membrane is clamped between the first screw body part and the second screw body part, either rigidly or with some play. The membrane is thus preferably held by the first and second screw body parts, wherein the two screw body parts are connected to each other by way of the second external thread and the internal thread. A perforation in the membrane in each case serves as an aperture for a respective bone screw.

The membrane can be a membrane that is known from the prior art and can be used for bone distraction or callus distraction. The membrane is therefore a distraction membrane. The membrane can be planar or arched.

In a preferred embodiment, the membrane has at least one perforation, and more particularly a plurality of perforations, preferably at least as many perforations as the device has bone screws. In a preferred embodiment, the membrane has at least four perforations. In a preferred embodiment, the diameters of the perforations correspond to the diameter of the second external thread, so that the second external thread can be passed through the perforations. In one embodiment, at least some of the perforations are circular. In a further embodiment, at least some of the perforations are elliptical. In a further embodiment, at least some of the perforations are designed as elongated holes. In a further embodiment, at least some of the perforations are designed as angular elongated holes. Of course, it is possible to combine round holes, elliptical holes, elongated holes and angular elongated holes on a membrane, so that a membrane can comprise at least one round hole, at least one elongated hole and/or at least one angular elongated hole. According to a preferred embodiment, at least two perforations are designed as circular holes and two perforations are designed as elongated holes. According to a further preferred embodiment, at least two perforations are designed as circular holes and two perforations are designed as elongated holes, wherein one of the elongated holes is an angular elongated hole. According to a further preferred embodiment, at least one perforation is designed as a circular hole and at least three perforations are designed as elongated holes. The elongated holes are preferably designed so that the widths of the elongated holes correspond to the diameter of the second external thread of a bone screw, and the lengths of the elongated holes are greater than the diameter of the second external thread, for example approximately twice as long as this diameter, and more particularly twice as long. It is also possible in one embodiment for at least some of the perforations to be ellipsoidal, wherein the diameter formed by the two semi-minor axes preferably corresponds to the diameter of the second external thread.

The perforations are preferably distributed over the surface of the membrane. In a preferred embodiment, in particular at least one perforation is provided in the vicinity of each corner of a rectangular membrane. As an alternative embodiment, the edges of the membrane body can comprise bulges, for example four bulges, each comprising a perforation.

The perforations designed as elongated holes, angular elongated holes or ellipses advantageously allow a bone screw, which extends through the corresponding perforation, to move along the length of the elongated hole or the major axis of the ellipsis. This is advantageous, for example, in the case of inclined atrophy of an alveolar ridge relative to the chewing plane, because here an oblique distraction can be carried out, wherein only some of the bone screws extending through the membrane, for example only two of the four bone screws, are slowly unscrewed from the bone, whereby the penetration positions of the bone screws through the membrane are varied slightly, which can be compensated for by the elongated holes or ellipses.

In a preferred embodiment, the membrane has a number of perforations that at least corresponds to the number of bone screws of the device.

In a preferred embodiment, the distraction membrane has a plurality of perforations. In a preferred embodiment, the diameters of the perforations are smaller than the diameter of the second external thread of the bone screws.

A diameter that is smaller than the diameter of the second external thread of a bone screw can advantageously be used to drill out the perforation to the size of the diameter of the second external thread. To this end, drilling can advantageously be carried perpendicular to the membrane or obliquely relative to the membrane. This can advantageously also be provided for in a kit according to the invention.

In an alternative, preferred embodiment, the diameters of the perforations correspond to the diameter of the second external thread. This allows a second external thread of a bone screw to be pushed directly through a perforation. In a further embodiment, the diameters of the perforations are minimally larger than the diameter of the second external thread. For example, the diameter of the perforation can be 5 to 15%, in particular approximately 10%, larger than the diameter of the second external thread. This allows a second external thread of a bone screw that is passed through such a perforation to have some play in the perforation, allowing an at least slightly oblique position of the bone screw relative to the membrane.

In a preferred embodiment, the membrane, also referred to as the distraction membrane, is suitable for callus distraction, especially in the jaw region, wherein the membrane comprises a contact surface and a counter-surface.

In a preferred embodiment, the membrane, also referred to as the distraction membrane, is suitable for callus distraction, especially in the jaw region, wherein the membrane comprises a contact surface and a counter-surface, and wherein at least one sub-region of the contact surface and at least one sub-region of the counter-surface are arched. The membrane is thus arched over at least a portion of the length or width of the membrane.

In a preferred embodiment, the membrane, also referred to as the distraction membrane, is suitable for callus distraction, especially in the jaw region, wherein the membrane comprises a contact surface, a counter-surface and at least one lateral face, and wherein the edges between the contact surface and the at least one lateral face and/or between the counter-surface and the at least one lateral face are rounded.

A preferred embodiment is a membrane, also referred to as a distraction membrane, that is suitable for callus distraction, especially in the jaw region, wherein the membrane according to the invention is arched, and wherein the membrane comprises a contact surface, a counter-surface and at least one lateral face, and wherein the edges between the contact surface and the at least one lateral face and/or between the counter-surface and the at least one lateral face are rounded.

In a preferred embodiment, the membrane, also referred to as the distraction membrane, is suitable for callus distraction, especially in the jaw region, wherein the membrane comprises a contact surface, a counter-surface and at least one lateral face, and wherein the at least one lateral face is bent, in particular bent toward the contact surface.

A preferred embodiment is a membrane, also referred to as a distraction membrane, that is suitable for callus distraction, especially in the jaw region, wherein the membrane according to the invention is arched, and wherein the membrane comprises a contact surface, a counter-surface and at least one lateral face, and wherein the at least one lateral face is bent, in particular bent toward the contact surface.

In the context of the present invention, a membrane is understood to mean a medical membrane, which is suitable for the distraction of a bone, preferably of a jaw bone, especially in the dental field. Such a membrane is also referred to as a distraction membrane.

The present teaching includes, in particular membranes, distraction devices and methods for bone regeneration, wherein preferably bones in the jaw region and/or in the periodontal region are to be regenerated.

In the present invention, the term ‘bone regeneration’ in particular is understood to also mean the regeneration of bone defects, for example after cystectomy, tumor surgery or trauma surgery or the like, regardless of the topography, and/or in particular also the regeneration of smaller bone defects caused by periodontitis, for example.

In the context of the present invention, a membrane is understood to mean a body that is plate-shaped, which is to say planar or flat, in the non-arched state. The membrane has a contact surface, which is used for osteoblasts to line or adhere to the region of a bone defect, and a counter-surface located opposite the contact surface. These two surfaces can take on any shape, for example they can be round, oval, quadrangular or polygonal. The contact surface and the counter-surface of the membrane are preferably rectangular in the non-arched state. In the non-arched state, the sizes of these two surfaces in a rectangular membrane result from the length and width of the membrane. The membrane additionally has at least one lateral face, and more particularly four lateral faces if it is a rectangular membrane. In the non-arched state, the sizes of two of the lateral faces result from the height and the length of a rectangular membrane, and the sizes of the two remaining lateral faces result from the height and width of the membrane. According to the invention, the membrane is as thin as possible, which means that the sizes of the lateral faces are several times smaller than the size of the contact surface, and in the case of a quadrangular membrane, the height of the membrane is several times smaller than the length and the width of the membrane.

A distraction membrane, which is suitable for bone distraction in the jaw region, is preferred, wherein the membrane comprises a contact surface and a counter-surface, and wherein at least a sub-region of the contact surface and a sub-region of the counter-surface are arched, and wherein the membrane has rounded edges.

A membrane that comprises a contact surface, a counter-surface and at least one lateral face is preferred, wherein at least one sub-region of the contact surface and a sub-region of the counter-surface are arched, and wherein the edges between the contact surface and the at least one lateral surface and/or between the counter-surface and the at least one lateral face are rounded.

In a preferred embodiment, the entire contact surface and the entire counter-surface are arched.

Arching in the context of the present invention shall be understood to mean a curvature of surfaces, in the present invention the contact surface and the counter-surface. According to the invention, the membrane is preferably singly arched, which in the case of a rectangular membrane means that two mutually opposing lateral faces are curved and the two other lateral faces are not curved.

In one embodiment, the membrane is designed as a shell, which is to say a membrane that is singly or doubly curved or arched.

In one embodiment, the membrane is arched so that it has the shape of a segment of a spherical shell, for example a hemispherical shell. In a further embodiment, the membrane is arched so that it has the shape of a cylindrical shell.

In a preferred embodiment, the radius of the arching corresponds to the radius of a bone to be treated, for example a long bone or a cranial bone.

In a preferred embodiment, the arching has a radius of at least 5 mm. In a preferred embodiment, the arching has a radius of no more than 15 mm. In a preferred embodiment, the arching has a radius of at least 5 mm and of no more than 15 mm.

In an alternative embodiment of the invention, the respective edges that are formed by two lateral faces can be rounded.

In a preferred embodiment, the membrane has rounded edges.

During the distraction of the membrane, the rounded edges and the arching of the membrane advantageously also prevent a sudden increase in pressure in the surrounding vessels, especially the smaller vessels. Preventing such a sudden rise in pressure is beneficial for wound healing.

A membrane that comprises a rectangular contact surface, a counter-surface and four rectangular lateral faces is preferred, wherein the contact surface and the counter-surface are arched, and wherein all the edges of the membrane are rounded.

In a likewise preferred alternative embodiment, the membrane is shaped and dimensioned so as to cover at least a portion of the surface of a jaw bone facing the teeth. A planar or an arched membrane can thus be provided, which in the planar state, which is to say in the flat state, is bent approximately in a horseshoe shape and has a length so that the membrane can cover at least a sub-region of an alveolar ridge. It is possible, in particular, for the membrane to cover the majority, for example up to 80%, of an alveolar ridge, or an entire alveolar ridge. A person skilled in the art, for example a dental technician, will be readily able to determine the size and shape of a membrane that is required to cover a desired alveolar ridge region. Membranes thus shaped can advantageously be used to treat wider bone defects, for example bone defects that cover several missing teeth, and even the entire alveolar ridge.

The shape and size of the membranes can be ready-made or individually adapted to the bone defected to be treated.

In a further embodiment, the membrane has at least one further arching, and more particularly several additional archings having smaller radii.

In a preferred embodiment, the membrane has bent edges. In a preferred embodiment, the membrane has at least two bent lateral faces.

In a preferred embodiment, the membrane has at least one perforation.

In a preferred embodiment, the membrane comprises titanium. In a preferred embodiment, the membrane is made of titanium. In a further embodiment, the membrane can also be made of a biodegradable material or comprise the same.

In a preferred embodiment, the membrane is sand-blasted. In a preferred embodiment, the contact surface of the membrane is sand-blasted.

In a preferred embodiment, the contact surface of the membrane is coated.

In a preferred embodiment, the edges of the membrane are covered with a non-woven fabric or a film.

The membrane according to the invention can be intended for multiple uses or for single use.

The membrane is preferably intended for single use because this is common practice with medical membranes, and the adhesive power of the surface of the membrane decreases upon contact with body fluid. The membrane according to the invention can be intended for single use in particular if the membrane was individually produced for a particular bone defect and/or if the membrane comprises biodegradable constituents, which break down on use.

In a preferred embodiment, the membrane according to the invention comprises at least one securing element. The at least one securing element is used to secure the membrane to at least one actuating element. A securing element can be a perforation, a eyelet or an anchor point, for example. The at least one securing element is preferably a perforation, and more particularly a perforation for inserting a screw.

The present invention also relates to a membrane according to the invention, wherein the membrane is secured to at least one actuating element. In one embodiment according to the invention, the actuating element is a screw. In an alternative embodiment, the membrane is secured to several actuating elements, in particular screws.

In a preferred embodiment, the second external thread of a respective screw body part of a bone screw according to the invention extends through a perforation in the membrane, wherein the second external thread of the screw body part is screwed to the internal thread of the other screw body part of the bone screw.

In a preferred embodiment, the second external thread of a respective first screw body part of a bone screw of the first embodiment extends through a perforation in the membrane, wherein the second external thread of the first screw body part is screwed to the internal thread of the second screw body part of the bone screw so that, at the edge of the hole, the membrane is clamped between the chamfered or rounded transition from the first external thread to the screw body intermediate piece of the first screw body part and the chamfer or rounding of the second screw body part that is directed toward the internal thread.

In a preferred embodiment, a respective second screw body part of a bone screw of the second embodiment extends through a hole in the membrane, wherein the second external thread of the second screw body part is screwed to the internal thread of the first screw body part of the bone screw so that, at the edge of the hole, the membrane is clamped between the chamfered or rounded end of the first screw body part and the chamfer or rounding of the screw head of the second screw body part that is directed toward the second external thread.

In a preferred embodiment, the membrane is not clamped fully between the first and second screw body parts, but has some play. In the context of the present invention, “clamped” shall be understood to mean that the membrane is either rigidly seated, which is to say has no play, or is seated with some play, which is to say the membrane can move to a small extent relative to a bone screw, between the two screw body parts. The freedom of movement referred to as play can refer to the longitudinal direction of the bone screw and/or to the transverse direction of the bone screw. The freedom of movement in the longitudinal direction of the bone screw can be varied by the length of the screw body intermediate piece compared to the thickness of the membrane. The freedom of movement in the transverse direction of the bone screw can be varied by the shape and the diameter of the perforation compared to the diameter of the screw body intermediate piece.

In a preferred embodiment, the at least two bone screws are not perpendicular to the membrane surfaces.

In the context of the present invention, ‘perpendicular to the membrane surfaces’ is understood to mean that the bone screw penetrates a perforation along an imaginary line through the perforation center on the outer surface of the membrane and the perforation center on the inner surface of the membrane. This also applies to curved membranes.

In an alternative embodiment, at least two bone screws are not perpendicular to the membrane surfaces, and at least two bone screws are perpendicular to the membrane surfaces. In an alternative embodiment, two bone screws are not perpendicular to the membrane surfaces, and two bone screws are perpendicular to the membrane surfaces.

In a preferred embodiment, at least some of the bone screws are located at an angle relative to the membrane surface, which deviates at least 0.1 degrees, and in particular at least 1 degree, from the perpendicular. In a preferred embodiment, at least some of the bone screws are located at an angle relative to the membrane surface, which deviates by no more than 25 degrees, and in particular by no more than 10 degrees, from the perpendicular.

It is also possible, in particular, for the membrane to be clamped by a bone screw so that play is possible, so that the screw extends through the membrane at an angle that deviates from the perpendicular by no more than 25 degrees, and in particular by no more than 10 degrees.

In a further embodiment, a fixation aid, and in particular a bioresorbable fixation aid, is associated with at least one, and more particularly with each, bone screw. The bioresorbable fixation aid can be a bone screw or a dowel, for example. The bone screw according to the invention can be screwed into the fixation aid by way of the first external thread, especially after the fixation aid has been introduced into a bone, in particular a jaw bone. In this embodiment using bioresorbable fixation aids, the device is preferably intended for single use.

In an alternative embodiment, the device according to the invention is a device for periodontal regeneration. Periodontal regeneration shall be understood to mean regeneration of the periodontium, which is to say not only of the bone, but also of the periodontal ligament, the periodontal tissue, the gingiva and the papilla, for example by way of guided tissue regeneration (GTR). In a preferred embodiment, the device comprises a membrane for periodontal regeneration. In a preferred embodiment, the membrane for periodontal regeneration has such small dimensions that the same can also be used in interdental spaces. In a preferred embodiment, the membrane for periodontal regeneration is very thin. In a preferred embodiment, the membrane for periodontal regeneration is shaped so that the membrane comprises at least one lobular extension or a segment that can be inserted into an interdental space. In a preferred embodiment, the membrane for periodontal regeneration is a single-piece, two-piece or multi-piece membrane. In a preferred embodiment, the membrane for periodontal regeneration comprises at least one securing element for securing a bone screw, for example at least one perforation. In a preferred embodiment, the bone screws of the device for periodontal regeneration are smaller than the bone screws of a device for bone regeneration.

A cable pull mechanism was known from the prior art from WO 01/91663 A1 as a device for periodontal regeneration. This mechanism is very complex, as a result of which it is not easy to use in the interdental region, and has unstable guidance.

An alternative embodiment of a device for periodontal regeneration is a periodontal membrane, through which the at least one bone screw extends, wherein the head of the bone screw is connected to a securing means. The membrane is moved along the screw body by turning of the bone screw. The bone screws are preferably resorbable screws for this purpose.

In a preferred embodiment, a bone screw according to the invention and/or a device according to the invention are intended for single use. It is common practice to use bone screws and a medical membrane, in particular a distraction membrane, only once, even if these are made of non-biodegradable material, such as titanium, because not only do these often need to be individually adapted, but the surface is also modified by the one-time use, so that further use would often take place under less than ideal conditions.

However, multiple uses can also be provided in an alternative embodiment.

A preferred embodiment is a device according to the invention for use with a medical indication, in particular with a first or a second medical indication. A preferred embodiment is a device according to the invention for medical use, and in particular surgical use. A further preferred embodiment is a device according to the invention for surgical use in the jaw region. A further preferred embodiment is a device according to the invention for use with callus distraction, especially in the jaw region.

A preferred embodiment of the bone screw according to the invention is such a bone screw for use for the production of a device for callus distraction, in particular in the jaw region, preferably for the production of a device according to the invention.

A preferred embodiment is the use of a bone screw according to the invention and/or of a device according to the invention in a medical procedure, in particular in a surgical procedure. A preferred embodiment is the use of a bone screw according to the invention and/or of a device according to the invention with callus distraction, especially in the jaw region.

A preferred embodiment is the use of a bone screw according to the invention and/or of a device according to the invention with periodontal regeneration.

The present invention also solves the underlying technical problem by a kit, including at least two bone screws according to the invention and at least one medical membrane, in particular a distraction membrane.

In a preferred embodiment, the kit includes granular bone substitute material as the fixation material for the bone screws.

In a preferred embodiment, the kit includes at least two bioresorbable fixation aids for the bone screws.

In a preferred embodiment, the kit includes a drilling template.

In a preferred embodiment, the kit includes instructions for use. The instructions for use preferably include descriptions of how the kit can be used to carry out a callus distraction.

In a preferred embodiment, the kit includes instructions for use. The instructions for use preferably include descriptions of how the kit can be used to carry out a periodontal regeneration.

The invention further relates in particular to a kit, including at least two bone screws, a distraction membrane and instructions for use, wherein the at least two bone screws are bone screws of the first embodiment or bone screws of the second embodiment, or some of the at least two bone screws are bone screws of the first embodiment and the remaining bone screws are bone screws of the second embodiment.

A preferred embodiment is a kit according to the invention for use in medical procedures, in particular surgical procedures, preferably during bone distraction, especially in the jaw region. A preferred embodiment is a kit according to the invention for use with periodontal regeneration. A further preferred embodiment is the use of a kit according to the invention for the production of a device according to the invention.

The present invention also solves the underlying technical problem by the use of a bone screw according to the invention, or a device according to the invention, or a kit according to the invention, for bone regeneration by way of distraction.

The present invention also solves the underlying technical problem by the use of a bone screw according to the invention, or a device according to the invention, or a kit according to the invention, for periodontal regeneration by way of distraction.

The present invention also relates to methods for callus distraction, and more particularly for reconstructing a jaw bone by way of callus distraction, using the device according to the invention, comprising the following steps:

a) screwing at least one first screw body part, and more particularly at least two or four, first screw body parts of bone screws according to the invention into a bone around a bone defect at a previously determined distance;
b) tightening a membrane by way of perforations of the membrane against the first screw body parts using the respective second screw body parts, so that the membrane covers the bone defect;
c) slowing unscrewing at least one, and more particularly at least two, for example, of all bone screws that have been screwed in.

The bone screws are preferably unscrewed at a rate at which the membrane moves away from the bone defect at a rate of at least 0.1 mm and no more than 2 mm per day. The rate is preferably approximately 0.1 mm to 2 mm per day, and more particularly 0.5 mm to 2 mm per day. However, the rate can also be approximately 1 mm per day.

Without being bound to theory, in particular, a distance of approximately 1.5 mm between the membrane and the bone is advantageous at the start of the procedure for such distraction methods.

Unscrewing can be carried out continuously or discontinuously, for example at daily or semi-daily intervals.

The present invention also relates to such methods for periodontal regeneration.

The invention and preferred embodiments of the invention will be described in greater detail hereafter based on the drawings. In the drawings:

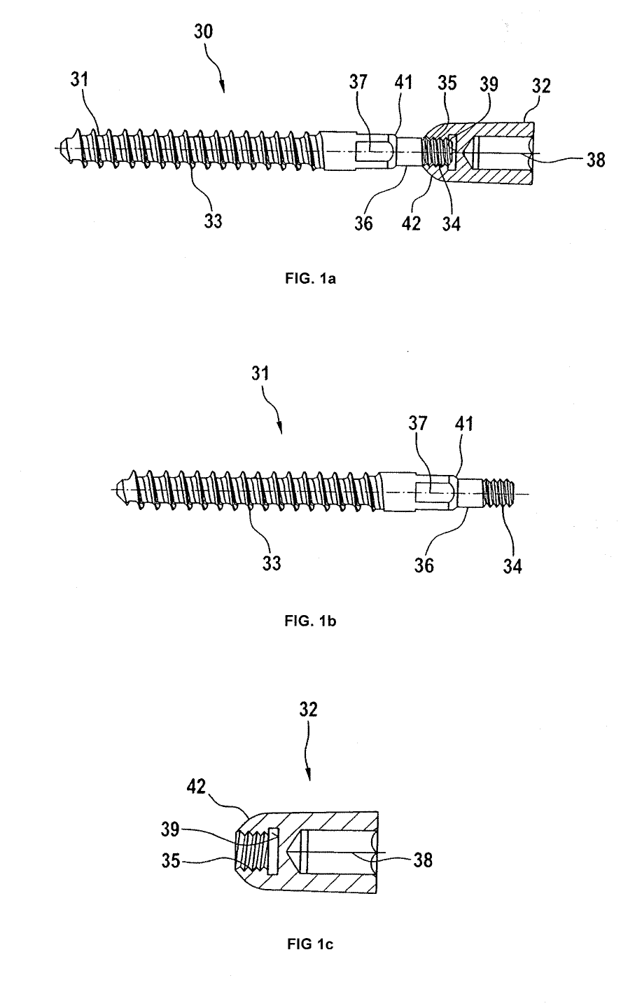

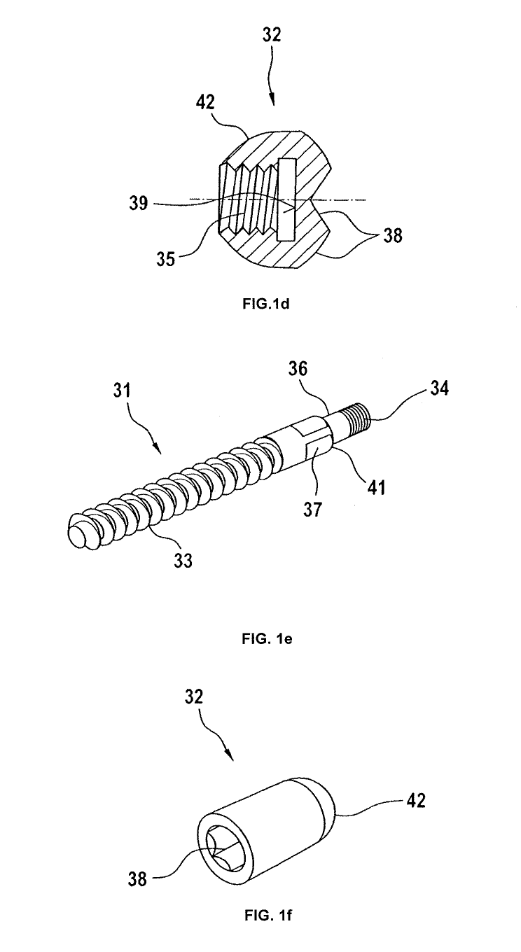

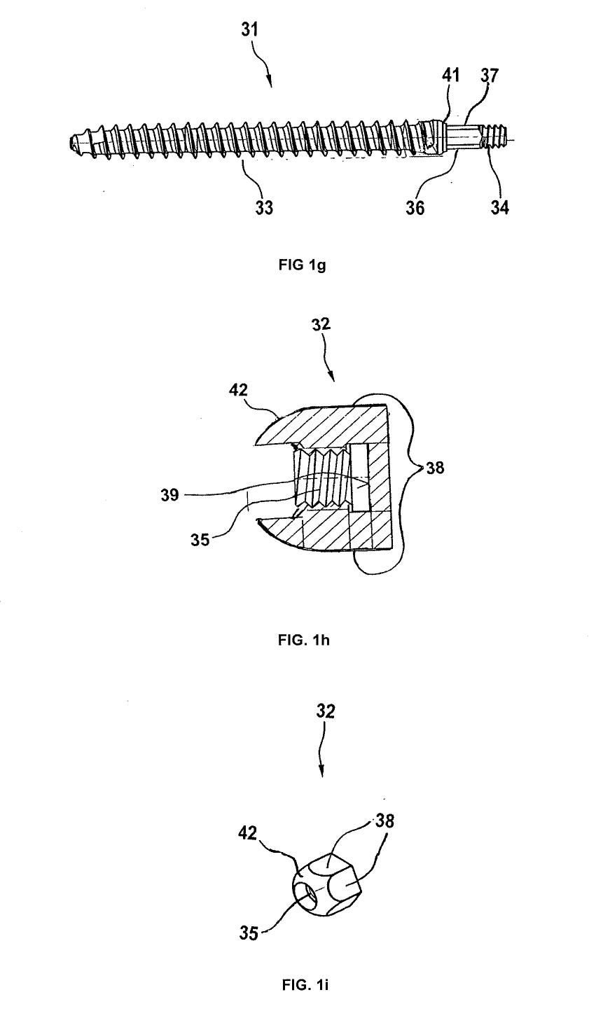

FIG. 1a shows a bone screw of the first embodiment;

FIG. 1b shows a first screw body part of a bone screw of the first embodiment;

FIG. 1c shows a second screw body part of a bone screw of the first embodiment;

FIG. 1d shows an alternative second screw body part of a bone screw of the first embodiment;

FIG. 1e shows a first screw body part of a bone screw of the first embodiment;

FIG. 1f shows a second screw body part of a bone screw of the first embodiment;

FIG. 1g shows an alternative embodiment of the first screw body part;

FIG. 1h shows an alternative second screw body part of a bone screw of the first embodiment;

FIG. 1i shows an alternative second screw body part of a bone screw of the first embodiment;

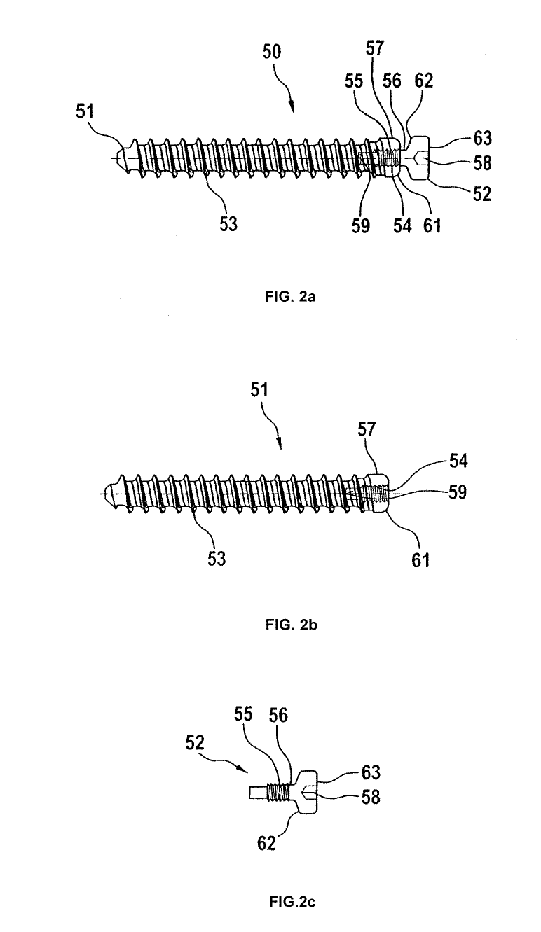

FIG. 2a shows a bone screw of the second embodiment;

FIG. 2b shows a first screw body part of a bone screw of the second embodiment;

FIG. 2c shows a second screw body part of a bone screw of the second embodiment;

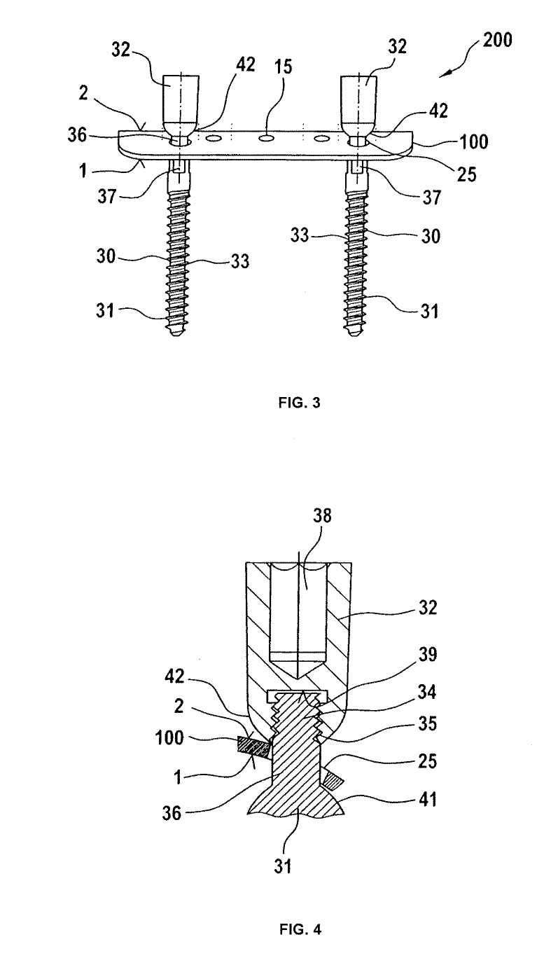

FIG. 3 shows a device according to the invention, comprising two bone screws of the first embodiment;

FIG. 4 is a detailed view of a device according to the invention, comprising a bone screw of the first embodiment;

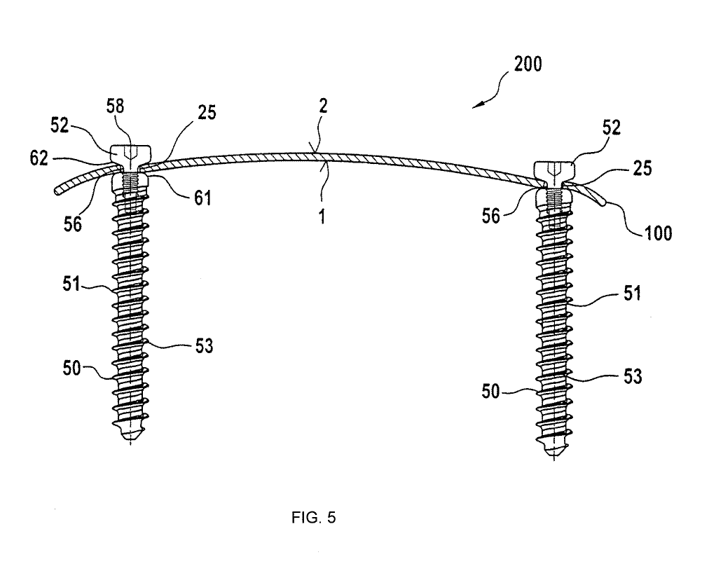

FIG. 5 shows a device according to the invention, comprising two bone screws of the second embodiment;

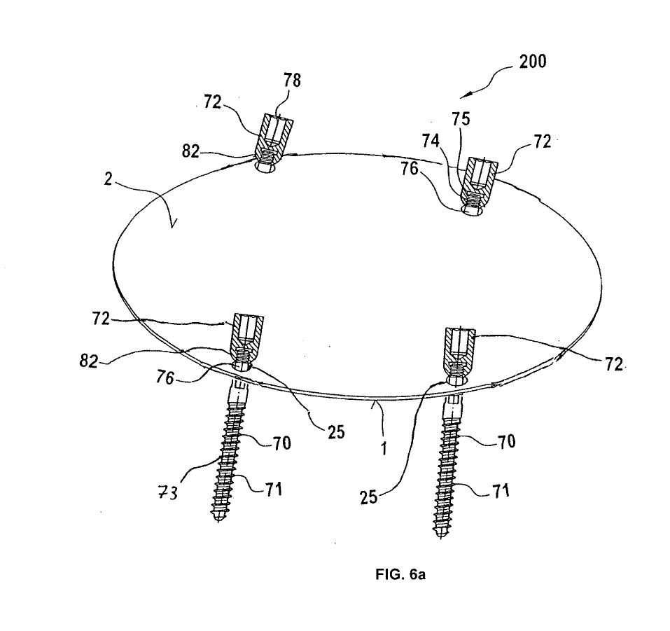

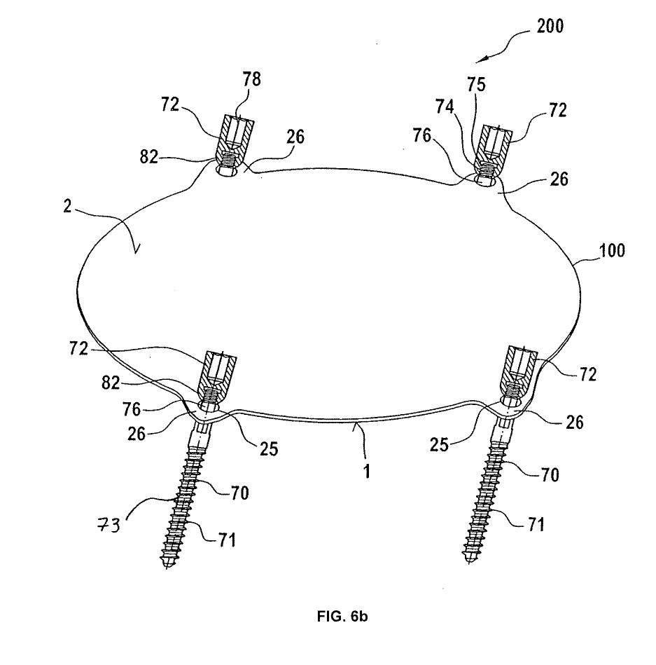

FIG. 6a shows a device according to the invention, comprising four bone screws;

FIG. 6b shows a device according to the invention, comprising four bone screws;

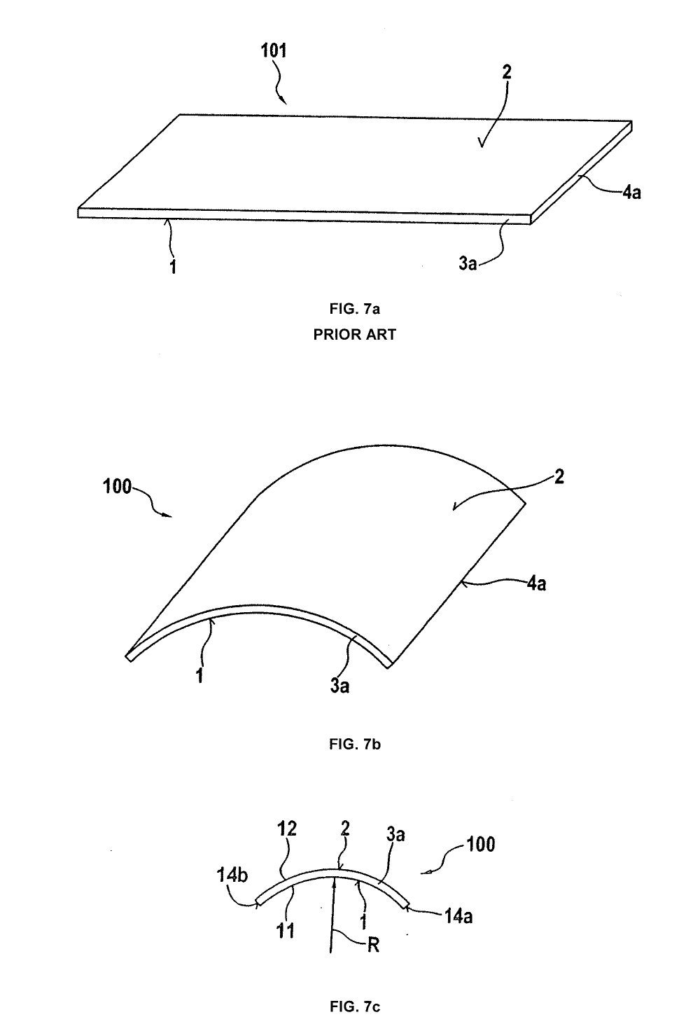

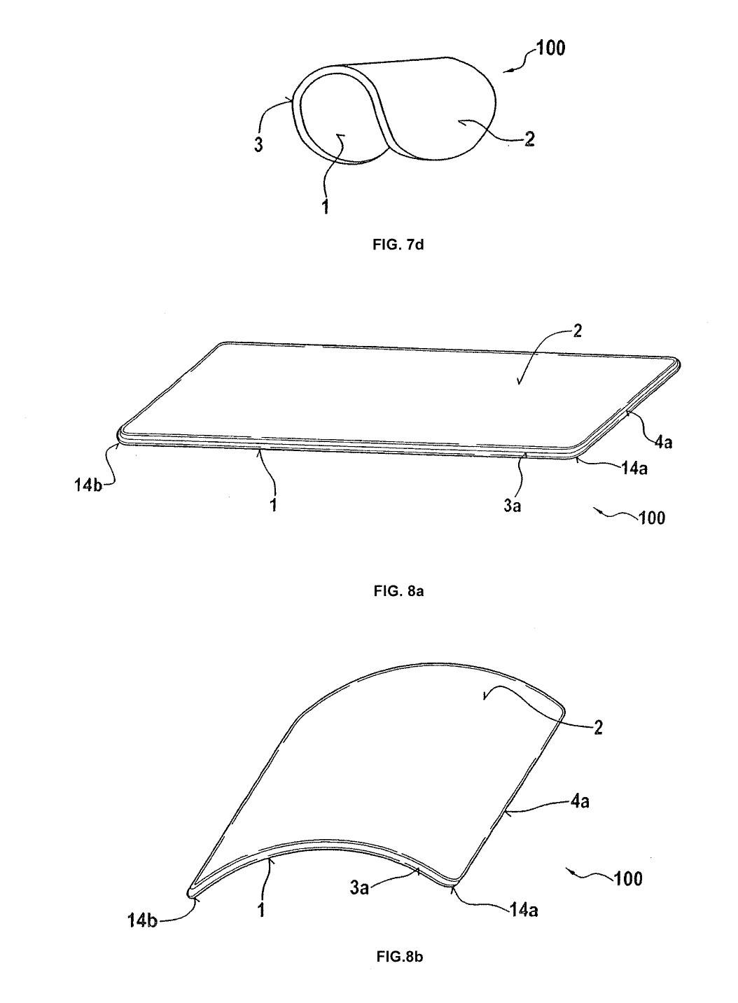

FIG. 7a shows a membrane of the prior art;

FIG. 7b shows a membrane according to the invention, comprising a rectangular contact surface;

FIG. 7c is a side view of a membrane according to FIG. 1b;

FIG. 7d shows a membrane according to the invention, comprising a circular contact surface;

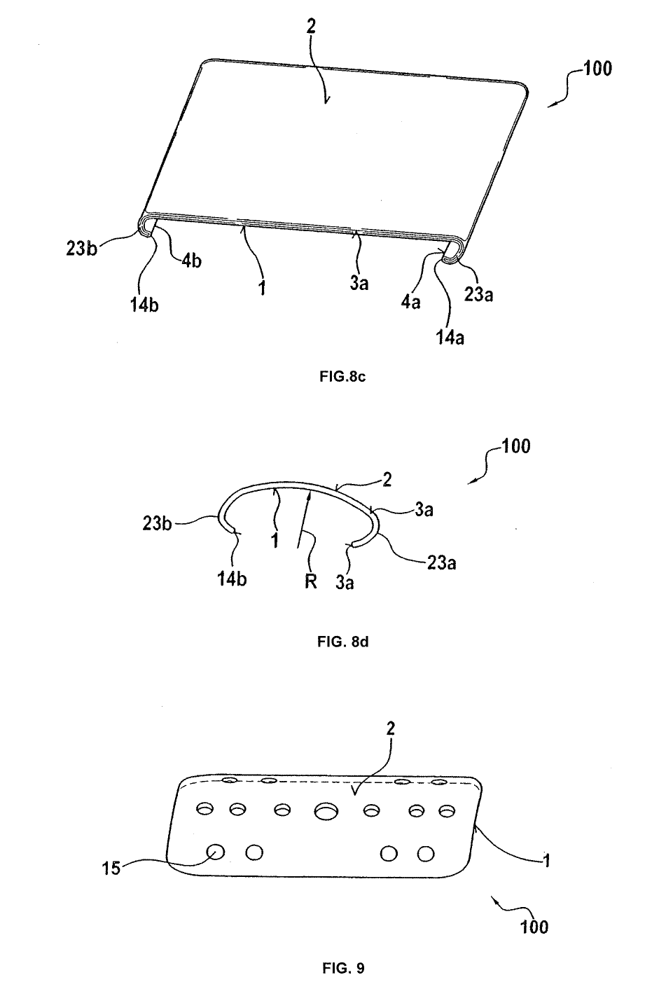

FIG. 8a shows a membrane according to the invention, comprising rounded edges;

FIG. 8b shows an arched membrane having rounded edges;

FIG. 8c shows a membrane having bent edge regions;

FIG. 8d shows an arched membrane having bent edge regions and rounded edges;

FIG. 9 shows an arched membrane having perforations;

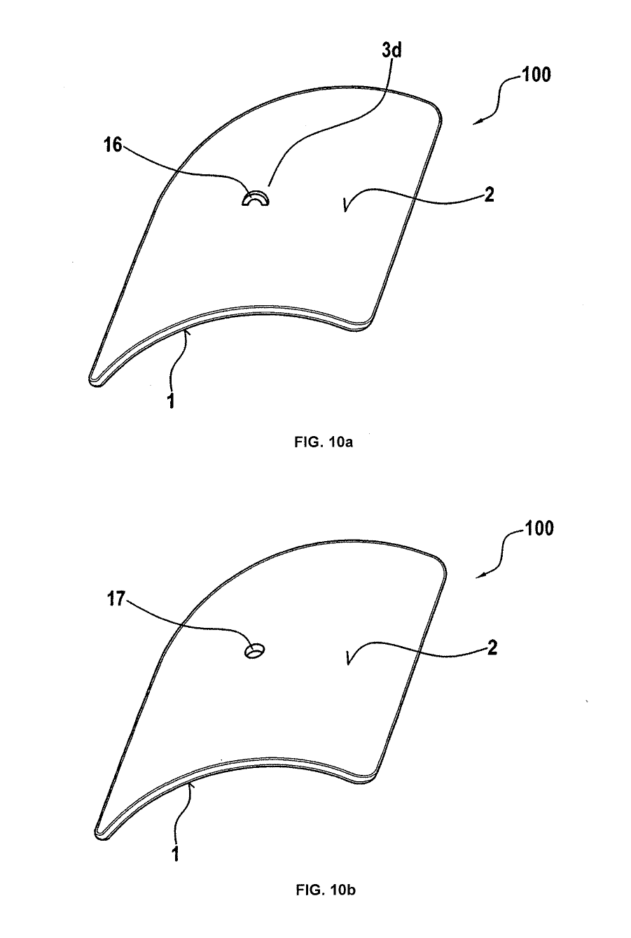

FIG. 10a shows a membrane comprising a eyelet as a securing element;

FIG. 10b shows a membrane comprising a hole as a securing element;

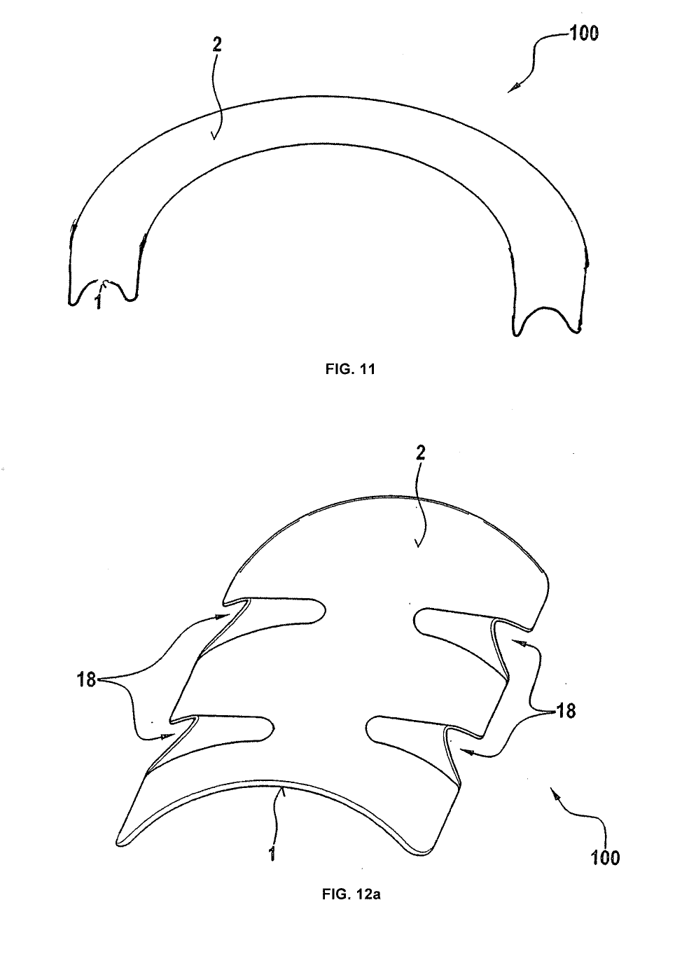

FIG. 11 shows a horseshoe-shaped membrane for treating large-surface-area jaw defects;

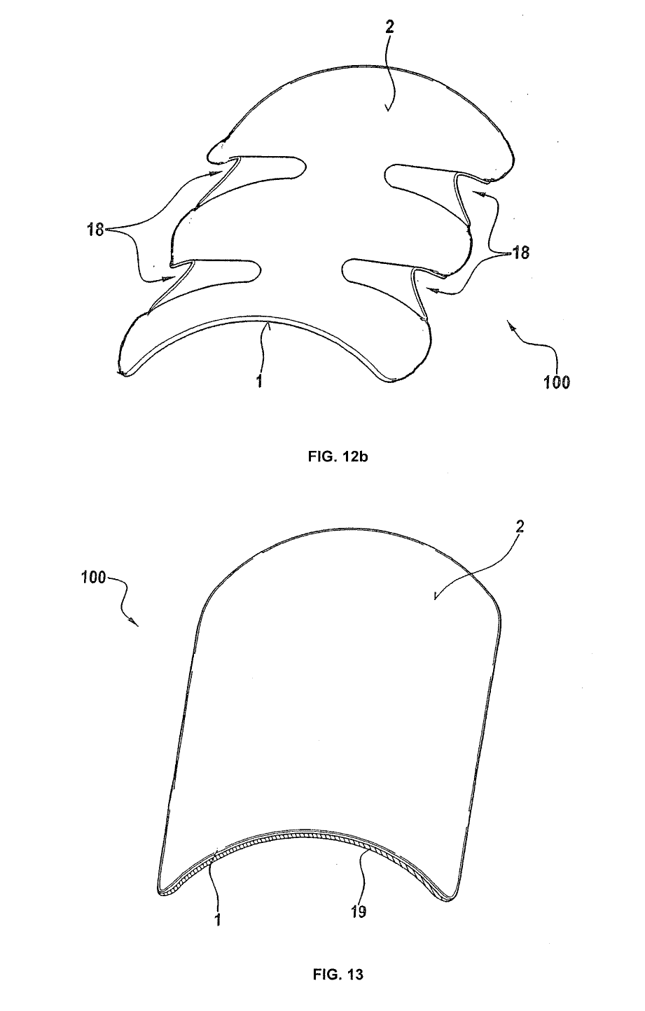

FIG. 12a shows a membrane comprising further archings for interdental papilla;

FIG. 12b is an alternative embodiment of the membrane comprising further archings for interdental papilla;

FIG. 13 shows a membrane, the contact surface of which is coated;

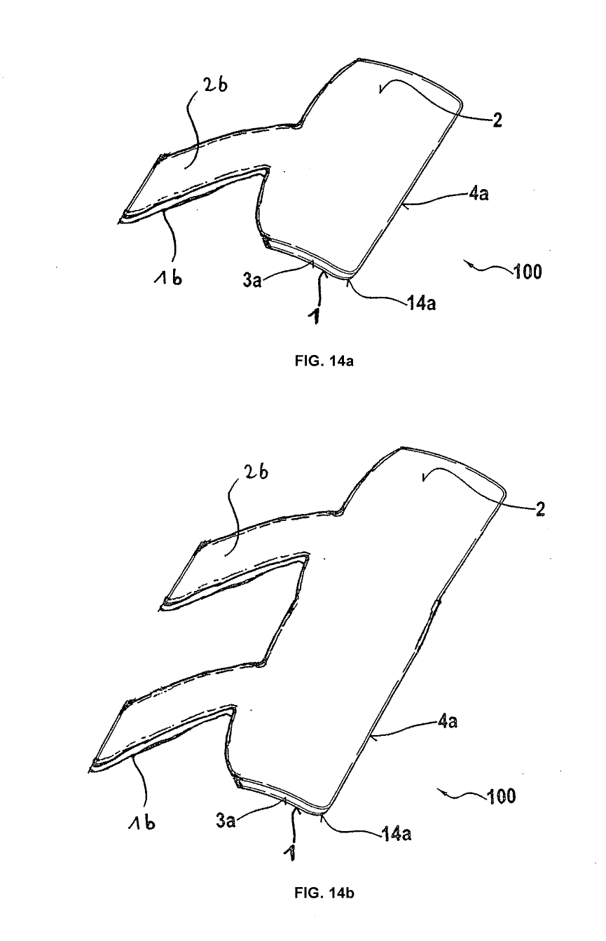

FIG. 14a shows a membrane for periodontal regeneration, comprising a segment for insertion into an interdental space;

FIG. 14b shows a membrane for periodontal regeneration, comprising two segments for insertion into an interdental space;

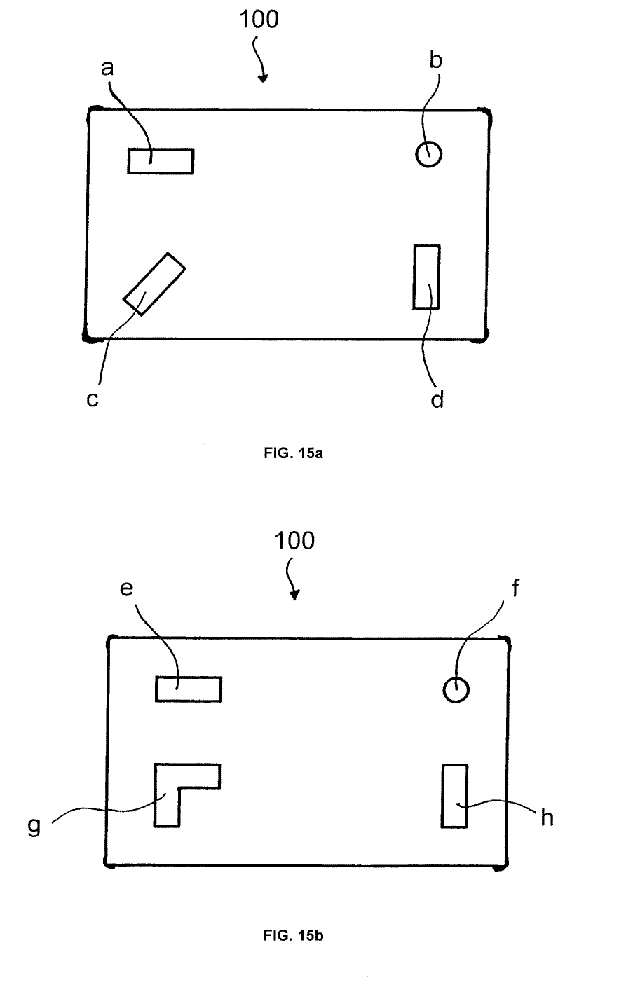

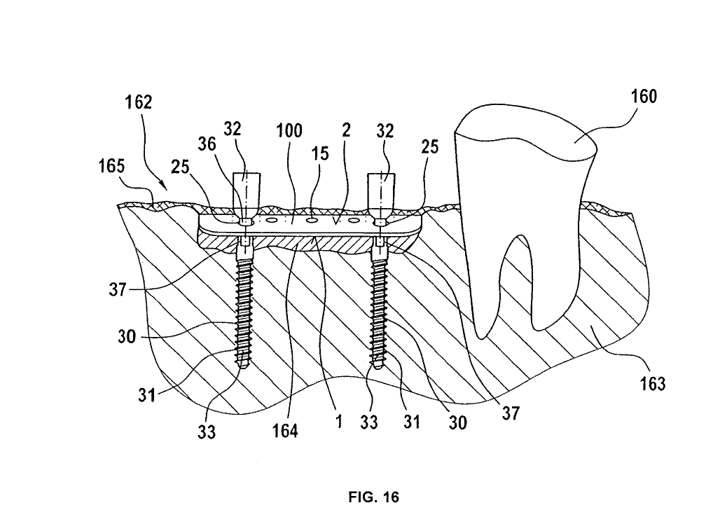

FIG. 15a shows a membrane comprising rounded edges and various perforations;

FIG. 15b shows a membrane comprising rounded edges and various perforations;

FIG. 16 shows a distraction device, comprising a membrane, a fixation device, and an actuating element connecting the fixation device to the membrane in the form of bone screws of the first embodiment; and

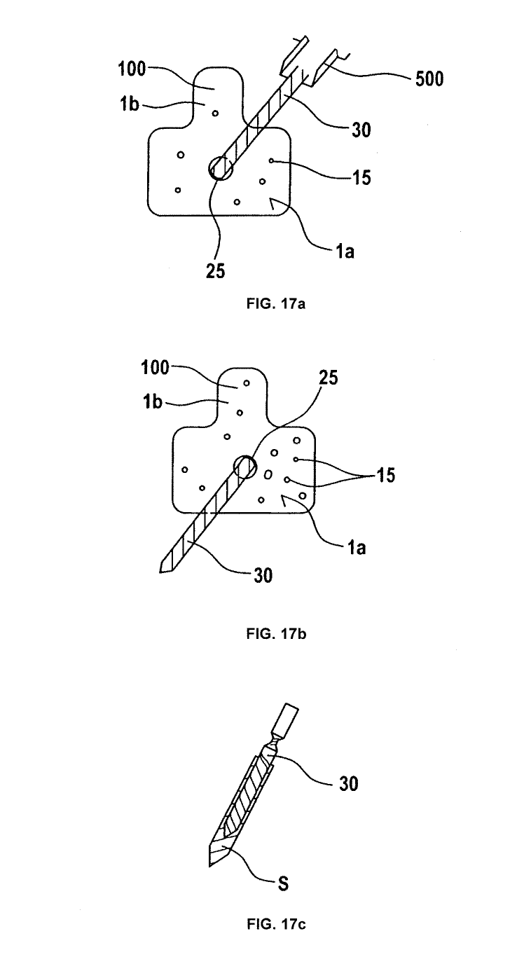

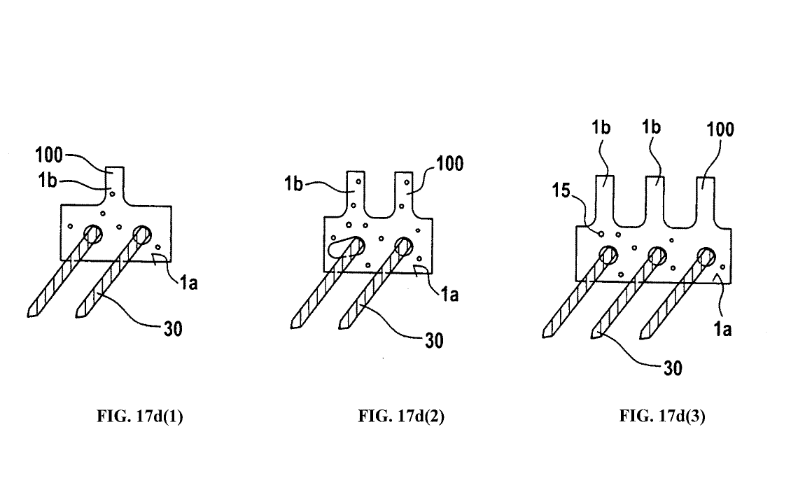

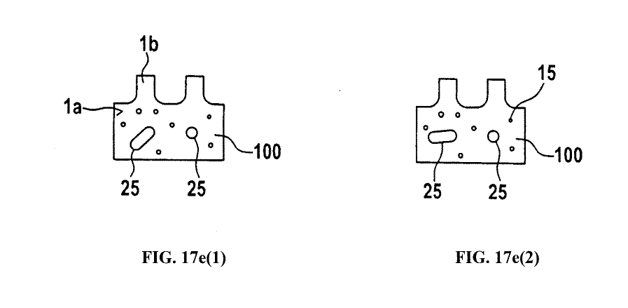

FIG. 17 shows examples of a device for periodontal regeneration.

FIG. 1a shows a bone screw of the first embodiment 30, comprising a first screw body part 31 and a second screw body part 32.

The first screw body part 31 comprises a right-handed external thread 33, which is used to fix the bone screw in a bone. The first screw body part 31 further comprises a section 37 for engaging a tool. The tool can be used to screw the screw into a bone. Because the section 37 directly follows the first external thread 33, the first screw body part 31 advantageously cannot be screwed too far into a bone by engaging a tool on the section 37 because the tool engaging on section 37 prevents continued turning by abutting upon the bone. The first screw body part 31 further comprises a screw body intermediate piece 36, which has a smaller diameter than the first external thread 33 and the section 37. The transition 41 from section 37 and the first external thread 33 to the screw intermediate piece 36 is rounded. A second external thread 34 follows the screw intermediate piece 36, this thread being oriented in the opposite direction of the first external thread 33, which is to say left-handed in the present case. The diameter of the second external thread 34 is at most as large as the screw body intermediate piece 36.

The bone screw 30 additionally comprises a second screw body part 32, which is provided with an internal thread 35, wherein the internal thread 35 is oriented in the same direction as the second external thread 34, this being left-handed in the present case. The second screw body part 32 has a rounded region 42 toward the opening of the internal thread 35. So as to screw the second body part 32 onto the first body part 31, the second body part 32 comprises an engagement section for a tool, which in the present case is a hex-head socket 38. The internal thread 35 advantageously protrudes into the second screw body part 32 far enough so that, when the second screw body part 32 is screwed onto a first screw body piece 31, the end face 39 delimiting the internal thread 35 limits the possibility of further turning due to contact with the second external thread 34, if the screw body intermediate part 36 between the rounded regions 41 and 42 has a desired length that approximately corresponds to the thickness of a membrane, so that the membrane is clamped between the first screw body part 31 and the second screw body part 32, and if desired is clamped therebetween with some play.

In FIG. 1a, the first screw body part 31 and the second screw body part 32 are connected to each other by way of the second external thread 34 and the internal thread 35. After the second screw body part 32 has been unscrewed from the first screw body part 31, a membrane having a perforation can be pushed past the second external thread 34 onto the first screw body part 31, so as to seat the membrane at the level of the screw body intermediate piece 36. The second screw body part 32 can then be screwed back on, whereby the membrane is clamped, optionally with some play, between the rounded regions 41 and 42 at the level of the screw body piece 36.

Of course, the first external thread 33 can also be left-handed, and the second external thread 34 and the internal thread 35 can thus be right-handed.

FIG. 1b shows a first screw body part 31 of a bone screw of the first embodiment. Again, the right-handed first external thread 33, the left-handed second external thread 34, the screw body intermediate piece 36, the section for engaging a tool 37 and the rounded transition 41 can be seen.

FIG. 1c shows a second screw body part 32 of a bone screw of the first embodiment. Again, the internal thread 35, the rounded region 42 at the opening of the internal thread 35 with the end face 39 delimiting the internal thread 35, and the engagement surface for a tool piece 38 can be seen.

FIG. 1d shows an alternative embodiment of the second screw body part 32. Again, the internal thread 35, the rounded region 42, the engagement surface for a tool 38, and the face 39 delimiting the internal thread toward the inside can be seen.

FIG. 1e shows the first screw body part 31, comprising the first external thread 33, the second external thread 34, the screw intermediate piece 36, the section for engaging a tool 37, and the rounded region 41, in a three-dimensional view.

FIG. 1f shows the second screw body part 32 comprising the rounded region 42 and the engagement surface for a tool 38, in a three-dimensional view.

FIG. 1g shows an alternative embodiment of the first screw body part 31, comprising the first external thread 33 and the second external thread 34. The screw intermediate piece 36 is also designed as a section for engaging a tool 37 and adjoins the rounded region 41.

FIG. 1h shows an alternative embodiment of the second screw body part 32. Again, the internal thread 35, the rounded region 42, and the face 39 delimiting the internal thread toward the inside can be seen. The engagement surface for a tool 38 is located on the outer surface of the screw body part 32. The internal thread 35 is slightly recessed inside the screw body part 32. This allows the slightly extended screw intermediate piece of FIG. 1g to be compensated for, which is also designed as a section for engaging a tool and is thus longer than the thickness of a distraction membrane.

FIG. 1i shows the second screw body part 32 of FIG. 1h, comprising the internal thread 35, the rounded region 42, and the engagement surface for a tool 38, in a three-dimensional view.

FIG. 2a shows a bone screw of the second embodiment 50, comprising a first screw body part 51 and a second screw body part 52.

The first screw body part 51 has a right-handed external thread 53, which is used to fix the bone screw in a bone. The first screw body part 51 further comprises a section 57 for engaging a tool. The tool can be used to screw the screw into a bone. Because the section 57 directly follows the first external thread 53, the first screw body part 51 advantageously cannot be screwed too far into a bone by engaging a tool on the section 57 because the tool engaging on section 57 prevents continued turning by abutting upon the bone. The first screw body part 51 further comprises an internal thread 54, which is oriented in the opposite direction of the first external thread 53, which is to say left-handed in the present case. The diameter of the internal thread 54 is smaller than the diameter of the first external thread 53. The first screw body part 52 has a rounded region 61 toward the opening of the internal thread 54. The internal thread 54 advantageously protrudes into the first screw body part 51 far enough so that, when a second screw body part 52 is screwed onto the first screw body part 51, the end face 59 delimiting the internal thread 54 limits the possibility of further turning due to contact with the second external thread 55, if the screw body intermediate piece 56 between the rounded regions 61 and 62 has a desired length that approximately corresponds to the thickness of a membrane, so that the membrane is clamped between the first screw body part 51 and the second screw body part 52, and if desired is clamped therebetween with some play.

The bone screw 50 additionally comprises a second screw body part 52, which is provided with a second external thread 55, wherein the second external thread 55 is oriented in the same direction as the internal thread 54, this being left-handed in the present case. The second screw body part 52 also comprises a screw body intermediate piece 56, the diameter of which is smaller than that of the first external thread 53. The transition 62 from the screw intermediate piece 56 to the head 63 of the second screw body part 52 is rounded. So as to screw the second body part 52 onto the first body part 51, the head 63 of the second body part 52 comprises an engagement section for a tool, which in the present case is a hex-head socket 58.

In FIG. 2a, the first screw body part 51 and the second screw body part 52 are connected to each other by way of the internal thread 54 and the second external thread 55. After the second screw body part 52 has been unscrewed from the first screw body part 51, a membrane having a perforation can be pushed past the second external thread 55 onto the second screw body part 52, so as to seat the membrane at the level of the screw body intermediate piece 56. The second screw body part 52 can then be screwed back on, whereby the membrane is clamped, optionally with some play, between the rounded regions 61 and 62 at the level of the screw body piece 56.

Of course, the first external thread 53 can also be left-handed, and the second external thread 55 and the internal thread 54 can thus be right-handed.

FIG. 2b shows a first screw body part 51 of a bone screw of the second embodiment. Again, the right-handed first external thread 53, the left-handed internal thread 54 comprising the end face 59 delimiting the Internal thread 54, the screw body intermediate piece 36, the section for engaging a tool 57, and the rounded region 61 at the opening of the internal thread can be seen.

FIG. 2c shows a second screw body part 52 of a bone screw of the second embodiment. Again, the second external thread 55, the screw body intermediate piece 56, and the head 63 comprising the rounded transition 62 and the engagement surface for a tool piece 58 can be seen.

FIG. 3 shows a device 200 according to the invention, comprising two bone screws 30 of the first embodiment. Both bone screws 30 comprise a first screw body part 31 and a second screw body part 32. Again, the right-handed first external thread 33, the screw body intermediate piece 36, the section for engaging a tool 37, and the rounded region 42 at the opening of the internal thread can be seen. The first screw body part 31 and the second screw body part 32 are screwed together by way of the second external thread and the internal thread, so that the second external thread and the internal thread cannot be seen.

By way of perforations 25, a membrane 100 is clamped between the first screw body part 31 and the second screw body part 32 at the level of the screw body intermediate piece 36 in a way that provides the membrane 100 with some play for movement on the longitudinal axis of the bone screws 30. The diameters of the perforations 25 are slightly larger than the diameter of the screw body intermediate piece 36, so that the membrane 100 also has some play in the transverse direction of the bone screws 30. Of course, the length and/or the diameter of the screw body intermediate piece 36 can be selected so that the membrane is clamped without play in the longitudinal direction and/or transverse direction of the bone screws 30. If the bone screws 30 are not at right angles relative to the membrane 100, for example because the membrane 100 is arched, as shown in FIG. 3, the contact surface 1 of the membrane 100 can be seated against the rounded region 41 (not shown) and/or the counter-surface 2 thereof can be seated against the rounded region 42.

The membrane 100 has additional perforations 15, which are used to exchange nutrients between the contact surface 1 and the counter-surface 2 of the membrane.

One embodiment of this device having very small dimensions, which are adapted to the region of two teeth, in particular to the interdental region, can be used for periodontal regeneration, for example.

FIG. 4 shows a detailed view of a device 200 according to the invention, comprising a bone screw 30 of the first embodiment. The bone screw 30 comprises a first screw body part 31 and a second screw body part 32 having a tool engagement surface 38. Shown is the second external thread 34, which is screwed to the internal thread 35, whereby the start of the second external thread 34 abuts upon the face 39 delimiting the internal thread 35. The screw body intermediate piece 36 runs obliquely through the perforation 25 of an arched membrane 100. As a result, sub-regions of the contact surface 1 of the membrane 100 are seated against the rounded transition 41, and sub-regions of the counter-surface 2 of the membrane 100 are seated against the rounded region 42 at the opening of the internal thread 35. The rounded regions 41 and 42 advantageously prevent the membrane 100 from becoming interlocked with or kinked by the bone screw 30.

FIG. 5 shows a device 200 according to the invention, comprising two bone screws 50 of the second embodiment. Both bone screws 50 comprise a first screw body part 51 and a second screw body part 52 having a tool engagement surface 58. Shown are the right-handed first external thread 53 and the screw body intermediate piece 56, which is delimited by the rounded regions 61 and 62. The first screw body part 51 and the second screw body part 52 are screwed together by way of the Internal thread 54 and the second external thread 55.

By way of perforations 25, a membrane 100 is clamped between the first screw body part 51 and the second screw body part 52 at the level of the screw body intermediate piece 56, in a way that provides the membrane 100 with almost no play for movement on the longitudinal axis of the bone screws 50. The diameters of the perforations 25 are slightly larger than the diameter of the screw body intermediate piece 56, so that the membrane 100 has some play in the transverse direction of the bone screws 50. Of course, the length and/or the diameter of the screw body intermediate piece 56 can be selected so that the membrane is clamped with or without play in the longitudinal direction and/or transverse direction of the bone screws 50. Because the bone screws 50 are not at a right angle relative to the membrane 100, because the membrane 100 is slightly arched, the contact surface 1 of the membrane 100 is seated against the rounded region 61 and the counter-surface 2 is seated against the rounded region 62.

FIG. 6a shows a device 200 according to the invention, comprising four bone screws 70. The bone screws 70 comprise a first screw body part 71 and a second screw body part 72 having a tool engagement surface 78. Shown are the first external thread 73 and the screw body intermediate piece 76, which is delimited by the rounded region 81 and the rounded region 82. The first screw body part 71 and the second screw body part 72 are screwed together by way of a screw connection 74/75 comprising an internal thread and the second external thread, this screw connection being oriented in the opposite direction of the first external thread 73.

By way of perforations 25, a membrane 100 is clamped between the first screw body parts 71 and the second screw body parts 72 at the level of the screw body intermediate piece 76. In preferred embodiments, the bone screws 70 can be bone screws of the first and/or of the second embodiments.

FIG. 6b shows a device 200 according to the invention as in FIG. 6a. However, the perforations 25 are provided on lobes 26 of the membrane 100.

FIG. 7a shows a distraction membrane 101 from the prior art. The distraction membrane 101 has a contact surface 1, which is not visible here, and a counter-surface 2, and of the four lateral faces, the faces 3a and 4a, which adjoin each other, can be seen. Such a membrane can be used as described in WO 01/91663 A1 and U.S. Pat. No. 5,980,252, wherein the contact surface 1 faces a bone and the membrane is moved away, for example pulled away, from the bone at a particular rate, for example approximately 1 mm per day, using a distraction device.

FIG. 7b shows a distraction membrane that is arched. The membrane 100 has a contact surface 1 and a counter-surface 2. In addition, the membrane has four lateral faces 3a, 3b, 4a and 4b, of which only the two mutually adjoining lateral faces 3a and 4a can be seen. In a preferred embodiment, the membrane is singly arched, as shown in FIG. 1b. According to the invention, the contact surface 1 is concavely curved and the counter-surface 2 is convexly curved. With a single curvature of the shown membrane 100 having rectangular faces, two mutually opposing lateral faces 3a and 3b are curved and the other two mutually opposing lateral faces 4a and 4b are not curved.

The arched geometry of the membrane advantageously results in greater stability of the membrane against warping. This makes it possible to use distraction membranes having a very small membrane height, which is to say membrane thickness. This is advantageous when using such a membrane for callus distraction in the jaw region, because here the membrane is placed under the mucous membrane, and membranes having a large height result in tension in the mucosal flaps, which can cause ischemia associated with tissue necroses. This can also result in membrane exposure, as a result of which a membrane that is subject to bacterial colonization has to be removed. The arched geometry of a membrane according to the invention now allows stable membranes having a low height to be used, so that tension on the mucous membrane can be avoided.

The membrane preferably has a height of no more than 1 mm, and more particularly 0.5 mm.

In a preferred embodiment, the membrane 100 has a length of at least 5 mm and no more than 120 mm and a width of at least 5 mm and no more than 120 mm. For example, the membrane can have a length of approximately 20 mm and a width of approximately 10 mm. The length and width information applies to the membrane in the non-arched state. The membrane can in particular have approximately the width of an alveolar ridge and the length of a portion of the alveolar ridge or of the entire alveolar ridge.

A wide variety of suitable materials for distraction membranes are known to a person skilled in the art. The membrane is preferably made of a biocompatible material. The membrane is preferably made of a metal, in particular titanium. Membranes made of metals such as titanium have the advantage that they are very stable, despite having a small height.

However, alternatively, the membrane can also be made of a biocompatible plastic material. The plastic material is preferably a bioresorbable plastic material. These have the advantage that they do not have to be removed after the distraction.

FIG. 7c shows a side view of the arched membrane 100 according to the invention. Shown is the curved lateral face 3a, the edge 11 of which adjoins the concave contact surface 1 and the curved edge 12 of which adjoins the convex counter-surface 2. The edges 14a and 14b of the lateral face 3a adjoin the lateral faces 4a and 4b.

The membrane 100 is arched evenly over the entire contact surface 1. However, it is also possible that only sub-regions of the contact surface 1 are arched, for example only the center third of the edge 11 is arched. The radius R of the arching can also be different in various regions of the contact surface. According to the invention, the radius R of the arching of the contact surface 1 is preferably adapted to the natural shape of a jaw. The radius of the arching of the contact surface is preferably at least 5 mm and no more than 15 mm, particularly preferably at least 5 mm and no more than 12 mm, and in particular at least 6 mm and no more than 10 mm. The radius R of the arching of the contact surface 1 is preferably at least 5 mm, particularly preferably at least 6 mm. The radius R of the arching of the contact surface 1 is preferably no more than 15 mm, particularly preferably 12 mm, and in particular no more than 10 mm. The radius R of the arching of the contact surface 1 is preferably approximately 6 to 7 mm.

An arched membrane according to the invention thus not only has the advantage of increased stability with a low height, but such a membrane is advantageously also shaped so that the arching is consistent with the anatomical and physiological conditions of the bone to be regenerated. This allows the bone to be regenerated over the entire contact surface of the membrane because this membrane, during distraction, has approximately the same distance from the regenerating bone at every point.

FIG. 7d shows an arched membrane 100 according to the invention, in which the contact surface 1 and the counter-surface 2 are not rectangular, but circular. Such a membrane thus has only one lateral face 3.

FIG. 8a shows a planar membrane 100 according to the invention, having rounded edges. A membrane on which all edges are rounded is preferred. However, it is also possible that only the edges between the contact surface 1 and the at least one lateral face 3a, 4a and/or the edges between the counter-surface 2 and the at least one lateral face 3a, 4a are rounded. Because the lateral faces 3a, 4a are very narrow due to the low height of the membrane, it is also possible for the edges between the contact surface 1 of the membrane 100 and the at least one lateral face 3a, 4a of the membrane, and between the counter-surface 2 of the membrane 100 and the at least one lateral face 3a, 4a of the membrane 100, to be rounded so that the two rounded regions transition into each other. It is thus possible for the at least one lateral face 3a, 4a to be rounded. This is shown in FIG. 2b. The edges 14a, 14b between the individual lateral faces 3a, 4a are also preferably rounded. Preferably not only the edges, but also the corners of the membrane are rounded.

Rounded region the edges of a membrane advantageously prevents the edges from injuring the surrounding tissue, for example by cutting or crushing the tissue or fine vessels and capillaries, when the membrane is moved during distraction. Because of the rounded edges and/or corners, a membrane according to the invention can advantageously slide past the adjacent tissue without damaging the same. The rounded edges advantageously make it easier to adapt the mucous membrane over the membrane.

FIG. 8b shows an arched membrane 100 having rounded edges 4a, 4b.

The combination of the arching according to the invention and rounded region of the edges according to the invention advantageously results in a membrane that protects the adjacent tissue particularly well during the distraction because the membrane edges do not compress the fine vessels and capillaries of the adjacent tissue, which are very important for providing tissue nutrients to the mucous membrane covering the membrane. This prevents premature membrane exposure.

According to a further embodiment, the edges can additionally be covered with a non-woven fabric or a film. The non-woven fabric or the film can be bioresorbable or non-bioresorbable. Covering the edges with a non-woven fabric or a film offers additional protection for the adjacent tissue, in particular if the membrane is made of a very hard material, such as titanium for example.

FIG. 8c shows a planar membrane 100 having rounded edges 14a, 14b, wherein the lateral faces 4a, 4b are also bent toward the contact surface 1. The membrane is thus bent in at least two outer regions of the contact surface 1 and of the counter-surface 2, in particular it is bent toward the contact surface 1.

Bending the at least one lateral face 4a, 4b of the membrane also protects the adjacent tissue when the membrane is used because the tissue is seated against the bend, and not against a sharp edge. The edges therefore do not necessarily have to be rounded when the lateral faces are bent.

Because, during use, the membrane 100 is typically moved in the direction of the counter-surface 2, the lateral faces 4a, 4b are preferably bent in the direction of the contact surface 1.

FIG. 8d shows an arched membrane 100 having rounded edges 14a, 14b, wherein the lateral faces 4a, 4b are also bent toward the contact surface 1. The bends 23a, 23b of the edges 4a, 4b can be seen here as amplified arches of the basic arching of the membrane 100. In a preferred embodiment, the bends thus have a radius that is smaller than the radius R of the basic arching of the membrane.

FIG. 9 shows an embodiment of an arched membrane 100, wherein the membrane 100 is perforated so that the contact surface 1 and the counter-surface 2 are connected to each other by at least one hole 15, and more particularly by a plurality of holes, which can be distributed in particular over the entirety of the surfaces. In a preferred embodiment, the perforation holes have a diameter at least approximately 0.3 mm and no more than approximately 1.3 mm.

Perforation holes, and perforation holes having a diameter of approximately 1 mm in particular, allow capillaries to grow through the membrane, whereby excellent blood circulation and immune defense are ensured in the region of the newly formed bone. The perforations allow good blood flow through the mucous membrane covering the membrane and through the regenerated tissue between the membrane and the bone.

The number of perforation holes preferably varies depending on the size of the membrane. For example, a membrane having a length of approximately 20 mm and a width of approximately 10 mm can have approximately 10 to 20 perforation holes. Such a ratio of the number of perforation holes to the membrane surface provides an optimum balance between the total hole surface promoting blood circulation and the adhesion surface for osteoblasts adhering to the membrane during the distraction process.

FIG. 10a shows a membrane 100 that is arched according to the invention, comprising a eyelet 16 as a securing element. The eyelet 16 is located on the counter-side 2 of the membrane 100. A distraction device can also be attached to the eyelet 16, for example by way of a wire. By virtue of the distraction device, the membrane 100 can be used for callus distraction in the defect region of a bone by being pulled away from a bone defect at an adapted rate of approximately 1 mm per day.

FIG. 10b shows a membrane 100 that is arched according to the invention, comprising a hole 17 as a securing element. For example, a toothed rack of a distraction device can be inserted into the hole 17 and secured to the membrane 100, for example by way of welding, in particular laser welding, soldering or gluing.

FIG. 11 shows a preferred horseshoe-shaped embodiment of the membrane 100 for treating large-surface-area jaw bone defects.

The membrane 100 shown can be used, for example, if all the teeth of a jaw are missing and the jaw bone has to be regenerated so as to be able to perform implants. If not all the teeth of the jaw are missing, but only a large number of teeth that are located next to each other, the membrane 100 can be shortened and adapted accordingly.

FIG. 12a shows an alternative embodiment of the membrane 100 according to the invention, comprising the contact surface 1 and the counter-surface 2, in which additional archings 18 for the interdental papilla are provided. These additional archings 18 are adapted to the shape of the jaw bone forming the interdental papilla. The jaw bone is raised between two adjacent teeth in a region of approximately 2 mm, whereby the periodontium is higher there than in the region of the teeth. The additional archings can be provided so as to preserve this raised bone area even after bone regeneration by way of distraction. In order for the archings to follow the shape of the interdental papilla, these preferably have a radius of 0.5 mm to 1.5 mm, in particular approximately 1 mm, and are located offset by an angle of approximately 90 degrees relative to the first arching according to the invention. In a planar membrane, these archings are located along the longitudinal sides of the membrane running parallel to the alveolar ridge. A person skilled in the art, for example a dental technician, will be able to determine without great effort the dimensioning and positioning of additional archings that follow the shape of interdental papilla.

FIG. 12b shows an alternative embodiment of the membrane 100 of FIG. 6a. In this embodiment, the membrane surfaces 1, 2 are extended downward further between the additional archings 18, so that the alveolar ridge can also be covered laterally by the membrane. FIG. 13 shows a preferred embodiment of the membrane 100, in which the contact surface 1 of the membrane 100 is covered by a coating 19.

FIG. 13 shows an alternative embodiment of the membrane 100, in which the contact surface 1 of the membrane 100 is covered by a coating 19.

In a preferred embodiment, the contact surface 1 of the membrane 100 is coated with hydroxylapatite. In a further preferred embodiment, the contact surface 1 of the membrane 100 is coated with a bone substitute material, in particular a natural or synthetic bone substitute material.

The bone substitute material is preferably a natural bone substitute material, for example made of the mineral component of bones, in particular autogenous, allogenic or xenogenic bone, for example animal bones, and bovine bones in particular. A suitable bone substitute material is Bio-Oss®, which is available from Geistlich, for example.

In a further preferred embodiment, the contact surface of the membrane is coated with a bone substitute material and a biodegradable glue, in particular a fibrin glue. The bone substitute material is preferably joined to the contact surface of the membrane by way of the fibrin glue. Such a coating advantageously allows the newly generated bone tissue to adhere to the membrane before and during the distraction because the bone tissue can adhere well to the biological substitute material. In addition, such a coating allows for easy detachment, in particular even autonomous detachment of the membrane from the newly formed bone after completion of the distraction, because the fibrin glue is biodegradable, and thus is biologically degraded and decomposed during, and in particular after, the distraction. The coating made of bone substitute material thus detaches from the contact surface of the membrane. The membrane can be removed without having to be separated further from the bone, and the bone substitute material can remain in the restored bone defect.

FIGS. 14a and 14b show preferred embodiments of an arched membrane 100 for periodontal regeneration. The membranes 100 are very thin and have rounded edges 3a, 4a. The membranes comprise segments having the surfaces 1b and 2b, which can be inserted into the interdental spaces. The membrane in FIG. 14a has one such segment, and the membrane in FIG. 14b has two such segments. Of course, it is also possible to provide more than two, for example three or four, segments. The membrane can be inserted both from the vestibular side and from the lingual side, or simultaneously from both sides. Such a membrane is preferably used together with bone screws.

All of the membranes shown in FIGS. 7 to 14 can be used in a device according to the invention.

FIGS. 15a and 15b show membranes 100 having rounded edges and various perforations a, b, c, d, e, f, g, h. The membrane 100 in FIG. 15a has one round hole b and three elongated holes a, c, d. The elongated holes a, c, d are oriented in the direction of the round hole b in terms of the longitudinal extensions thereof. The membrane 100 in FIG. 15b has one round hole f, two elongated holes e, h, and one angular elongated hole g. The elongated holes e, h are oriented in the direction of the round hole f in terms of the longitudinal extensions thereof. Of course, the membrane 100 can also be arched and/or coated.

Membranes having such perforations or similarly shaped perforations for guiding through bone screws, in particular bone screws according to the invention, result in preferred devices according to the invention, in which a combined movement of the membrane away from the bone about several axes at one time is possible. This can be achieved by unscrewing the bone screws extending through the perforations from the bone at differing degrees each day. For example, bone screws extending through the perforations a and c can be unscrewed from the bone 1 mm per day, and bone screws extending through the perforations b and d can be unscrewed 0.5 mm per day. The bone screw extending through round hole b also may not be unscrewed at all, for example, whereby the round hole b forms the rotational axis of the membrane movement. The use of elongated holes can prevent the membrane from canting or jamming because the bone screws can slide in the elongated holes.

Of course, the preferred embodiments shown in FIGS. 1 to 15 can be arbitrarily combined with each other.

The present invention will be described in greater detail based on the following example and FIGS. 16 and 17,

FIG. 16 shows a distraction device 200, comprising an arched membrane 100 according to the invention and two bone screws 30 of the first embodiment. Both bone screws 30 comprise a first screw body part 31 and a second screw body part 32. Again, the right-handed first external thread 33, the screw body intermediate piece 36, and the section for engaging a tool 37 can be seen. The first screw body part 31 and the second screw body part 32 are screwed together by way of the left-handed second external thread and the left-handed internal thread, so that the second external thread and the internal thread cannot be seen. By way of perforations 25, the arched membrane 100 is clamped between the first screw body part 31 and the second screw body part 32 at the level of the screw body intermediate piece 36. The membrane 100 has additional perforations 15, which are used to exchange nutrients between the contact surface 1 and the counter-surface 2 of the membrane.

The respective first right-handed external thread 33 of the bone screws 30 is screwed into the jaw bone 163 in the region of a bone defect 162 next to a tooth 160. Screwing the screws in too far can advantageously be prevented because a tool, which engages on section 37 that directly adjoins the first external thread 33, is prevented from being turned further by the bone 163. The first screw body part 31 can preferably be inserted into the bone by way of a ratchet using a predefined torque. The ratchet has a socket that is specifically adapted to the section for engaging a tool 37. The bone defect 162 can be refreshed before or after screwing in the first screw body parts 31. The perforations 25 of the membrane 100 can be placed on the second external threads 34, and the second screw body parts 32 can be screwed on with the internal thread 35. The contact surface 1 of the membrane 100 is seated against the jaw bone in the bone defect. Because the membrane 100 approximately follows the arching of the jaw bone 163, the contact surface 1 is seated thereon evenly. Without being bound to theory, in particular a distance of approximately 1.5 mm between the membrane 100 and the bone 163 is advantageous. A coagel 164 has formed between the jaw bone 163 and the membrane 100, with osteoblasts also being present in this coagel. These osteoblasts can adhere to the contact surface 1 of the membrane 100. If the bone screws 30 are now turned counterclockwise by way of the second screw body parts 32, for example using a regular wrench or Allen wrench or the like, the bone screws 30 move out of the bone and upward, together with the membrane 100. Because the second external thread 34 and the internal thread 35 are left-handed, the first and second screw body parts 31/32 remain rigidly connected to each other when unscrewing a bone screw 30 from the bone. The bone screws 30 can be turned so that the membrane 100 moves away from the jaw bone 163 at a rate of approximately 1 mm per day. For example, the bone screws 30 can be turned once a day by a particular revolution, or twice a day by half this revolution. By lifting the membrane, biomechanical stimuli are applied to the osteoblasts adhering to the contact surface 1 in the coagel 164, resulting in osteogenesis. Using the perforations 15 in the membrane, the coagel 164 and the bone callus having formed therein are provided with sufficient circulation via the tissue 165 located over the membrane 100. Because of the arching of the membrane 100, the bone defect 162 of the jaw bone 163 is thus filled with new bone substance, so that the filled-in bone defect has the convexly shaped arching of the jaw bone 163.

FIG. 17 shows examples of membranes and devices for periodontal regeneration. The membranes 100 can advantageously be inserted both from the vestibular side and from the lingual side, or simultaneously from both sides. The base surface 1a of the membrane 100 covers approximately half the alveolar ridge. The segments 1b can be inserted into interdental spaces. The membranes can comprise a varying number of segments 1b, for example one, two, three or more segments, depending on the length of the membrane and depending on the number of interdental spaces to be treated. The membranes 100 have perforations 25, through which the bone screws 30 extend. The perforations can be designed as round holes, elongated holes, ellipses or angular elongated holes. The membranes may have further perforations 15, which are used to exchange nutrients. The bone screws are composed of two pieces. The two pieces are connected to each other by way of a thread, which, as with the bone screws according to the invention, is oriented in the opposite direction (for example a left-handed thread) of the thread proper of the bone screw (for example a right-handed thread). The bone screws can be accommodated in a larger bioresorbable screw S, which assures greater retention of the anchoring in the bone. The bioresorbable screw likewise has a thread (for example a left-handed thread), which is oriented in the opposite direction of the thread proper of the bone screw (for example a right-handed thread). Depending on the size of the membrane 100, this can be fixed by one, two, three or more bone screws. The screw head of a bone screw can be anchored in a securing means 500. As an alternative, it is also possible for the screw to be screwed through the securing means 500. The first screw body part can thus be screwed into a bone, and the second screw body part can be screwed into a securing means. FIG. 17a shows a device comprising a membrane 100, a screw 30, and securing means 500 from the side of the membrane that does not face the bone (counter-surface of the membrane). FIG. 17b shows the same device from the side that faces the bone (contact surface of the membrane). FIG. 17c shows a two-piece bone screw 30 according to the invention in a bioresorbable screw S. The bone screw 30 is preferably a bone screw according to the invention, in particular of the first embodiment, in particular in a miniaturized embodiment. FIG. 17d shows devices comprising membranes 100 that have a varying number of segments 1b. FIG. 17e shows membranes 100 having differing perforations 25, for example elongated holes and round holes, which are preferably arranged on the membrane.

It is a matter of course that the preferred embodiments shown in FIGS. 1 to 17 can be arbitrarily combined with each other.

1. A bone screw comprising a first screw body part and a second screw body part, wherein the first body part has a first external thread for sinking the screw into a bone and a second external thread that is oriented in the opposite direction of the first external thread, wherein the second external thread has a smaller diameter than the first external thread, and wherein a screw body intermediate piece is located between the first and second external threads, wherein the screw body intermediate piece has a diameter that is smaller than the diameter of the first external thread and at least equal in size to the diameter of the second external thread, and wherein a transition from the first external thread to the screw body intermediate piece has a chamfer or a rounding, and wherein the second screw body part is a screw nut comprising an internal thread, wherein the internal thread is oriented in the same direction as the second external thread of the first screw body part, and the first and second screw body parts can be detachably connected to each other by way of the second external thread and the internal thread, wherein the second screw body part has a chamfer or a rounding toward the opening of the internal thread, the opening facing the first screw body part when the parts are connected.

2. A bone screw comprising a first screw body part and a second screw body part, wherein the first body part comprises a first external thread for sinking the screw into a bone and an internal thread that is oriented in the opposite direction of the first external thread, wherein, at an end of the first screw body part located opposite a tip of the first screw body part, the internal thread extends partially into the first screw body part, and wherein the end of the first screw body part located opposite the tip of the first screw body part is chamfered or rounded, and wherein the second screw body part comprises a second external thread and a screw head that is chamfered or rounded toward the second external thread, wherein the second external thread is oriented in the same direction as the internal thread of the first screw body part, and the first and second screw body parts can be detachably connected to each other by way of the internal thread and the second external thread.

3. A device for bone distraction, comprising at least one bone screw according to claim 1 and a distraction membrane.

4. The device for bone distraction according to claim 3, wherein the distraction membrane comprises at least one perforation and wherein the diameters of the perforations are smaller than the diameters of the second external threads of the bone screws.

5. The device for bone distraction according to claim 3, wherein the second external thread of a respective first screw body part of the bone screw extends through a hole in the membrane, and the second external thread of the first screw body part is screwed to the internal thread of the second screw body part of the bone screw so that, at the edge of the hole, the membrane is clamped between the chamfered or rounded transition from the first external thread to the screw body intermediate piece of the first screw body part and the chamfer or rounding of the second screw body part that is directed toward the internal thread.

6. A device for bone distraction comprising at least one bow screw according to claim 2 and a distraction membrane, wherein a respective second screw body part of the bone screw extends through a hole in the membrane, and the second external thread of the second screw body part is screwed to the internal thread of the first screw body part of the bone screw so that, at the edge of the hole, the membrane is clamped between the chamfered or rounded end of the first screw body part and the chamfer or rounding of the screw head of the second screw body part that is directed toward the second external thread.

7. The device for bone distraction according to claim 5, comprising at least two of the bone screws, and wherein the bone screws not perpendicular to surfaces of the membrane.

8. A kit, comprising at least two bone screws according to claim 1 and at least one medical membrane.

9. The kit according to claim 8, further comprising granular bone substitute material as a fixation material for the bone screws.

10. The kit according to claim 8, comprising at least two bioresorbable fixation aids for the bone screws.

11. A device for bone distraction, comprising at least one bone screw according to claim 2 and a distraction membrane.

12. A device for bone distraction, comprising at least two bone screws according to claim 1 and a distraction membrane.

13. A device for bone distraction, comprising at least two bone screws according to claim 2 and a distraction membrane.

14. A device for bone distraction, comprising at least four bone screws according to claim 1 and distraction membrane.

15. A device for bone distraction, comprising at least four bone screws according to claim 2 and distraction membrane.

16. The device for bone distraction according to claim 11, wherein the distraction membrane comprises at least one perforation and wherein the diameters of the perforations are smaller than the diameters of the second external threads of the bone screws.

17. The device for bone distraction according to claim 12, wherein the distraction membrane comprises at least one perforation and wherein the diameters of the perforations are smaller than the diameters of the second external threads of the bone screws.

18. The device for bone distraction according to claim 13, wherein the distraction membrane comprises at least one perforation and wherein the diameters of the perforations are smaller than the diameters of the second external threads of the bone screws.

19. The device for bone distraction according to claim 14, wherein the distraction membrane comprises at least one perforation and wherein the diameters of the perforations are smaller than the diameters of the second external threads of the bone screws.

20. The device for bone distraction according to claim 15, wherein the distraction membrane comprises at least one perforation and wherein the diameters of the perforations are smaller than the diameters of the second external threads of the bone screws.

21. The device for bone distraction according to claim 5, wherein the distraction membrane comprises at least one perforation and wherein the diameters of the perforations are smaller than the diameters of the second external threads of the bone screws.

22. The device for bone distraction according to claim 6, comprising at least two of the bone screws, and wherein the bone screws are not perpendicular to surfaces of the membrane.

23. A kit, comprising at least two bone screws according to claim 2 and at least one medical membrane.

24. The kit according to claim 24, further comprising granular bone substitute material as a fixation material for the bone screws.

25. The kit according to claim 24, further comprising at least two bioresorbable fixation aids for the bone screws.
